(19) United States
(12) Patent Application Publication
(10) Pub. No.: US 2014/0170660 A1
(43) Pub. Date: Jun. 19, 2014

(54) METHODS AND COMPOSITIONS FOR PREDICTING UNOBSERVED PHENOTYPES (PUP)

(71) Applicant: Syngenta Participations AG, Basel (CH)

(72) Inventors: Zhigang GUO, Research Triangle Park, NC (US); Venkata Krishna KISHORE, Bloomington, IL (US)

(73) Assignee: Syngenta Participations AG, Basel (CH)

(21) Appl. No.: 14/186,473

(22) Filed: Feb. 21, 2014

Related U.S. Application Data
(63) Continuation of application No. 12/793,550, filed on Jun. 3, 2010.

(51) Int. Cl. C12Q 1/68

(52) U.S. Cl. CPC C12Q 1/6895
USPC 435/6.11

(57) ABSTRACT

Methods for predicting unobserved phenotypes are provided. In some embodiments, the methods include (a) determining marker effects for a plurality of markers in a genotyped and phenotyped reference population with respect to a phenotype, wherein the reference population includes an F₂ generation, an F₃ generation, or a subsequent generation; (b) genotyping one or more plants of a predicted population with respect to the plurality of markers, wherein each of the one or more plants of the predicted population is a descendant of two parents and each parent has at least 80% genetic identity to at least one of the two parental plants employed to generate the reference population; (c) summing the marker effects determined in step (a) for each of the one or more plants of the predicted population based on the genotyping of step (b); and (d) predicting a phenotype of the one or more plants of the predicted population based on the sum of the marker effects from step (c). Also provided are methods for generating a plant with a phenotype of interest, and methods for estimating genetic similarity between populations.

### CROSS-REFERENCE TO RELATED APPLICATIONS

This application is a continuation application of U.S. patent application Ser. No. 12/793,550 filed Jun. 3, 2010.

### TECHNICAL FIELD

The presently disclosed subject matter relates to molecular genetics and plant breeding. In some embodiments, the presently disclosed subject matter relates to methods for predicting unobserved phenotypes for quantitative traits using genome-wide markers across different breeding populations.

### BACKGROUND

A goal of plant breeding is to combine, in a single plant, various desirable traits. For field crops such as corn, these traits can include greater yield and better agronomic quality. However, genetic loci that influence yield and agronomic quality are not always known, and even if known, their contributions to such traits are frequently unclear.

Once discovered, however, desirable genetic loci can be selected for as part of a breeding program in order to generate plants that carry desirable traits. An exemplary approach for generating such plants includes the transfer by introgression of nucleic acid sequences from plants that have desirable genetic information into plants that do not by crossing the plants using traditional breeding techniques.

Desirable loci can be introgressed into commercially available plant varieties using marker-assisted selection (MAS) or marker-assisted breeding (MAB). MAS and MAB involve the use of one or more of the molecular markers for the identification and selection of those plants that contain one or more loci that encode desired traits. Such identification and selection can be based on selection of informative markers that are associated with desired traits.

However, even when the traits are known and suitable parental plants carrying the traits are available, producing progeny plants that have desirable combinations of the genetic loci associated with the traits can be a very long and expensive process. Typically, extensive breeding programs that can be very time consuming are required to produce progeny plants, each of which must be individually tested for the presence of the trait(s) of interest. This often also requires that the plants be allowed to grow to maturity since many if not most agriculturally important traits are ones that are displayed by mature plants as opposed to seedlings.

What are needed, then, are new methods and compositions for genetically and phenotypically analyzing plants, and for employing the information obtained for producing plants that have traits of interest.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The presently disclosed subject matter provides methods for predicting phenotypes in plants of predicted populations. In some embodiments, the methods comprise (a) determining marker effects for a plurality of markers in a genotyped and phenotyped reference population with respect to a phenotype, wherein the reference population comprises (i) an F₂ generation produced by crossing two parental plants to produce an F₁ generation and then intercrossing, backcrossing, and/or selfing the F₁ generation; and/or making a double haploid from F₁; and/or (ii) an F₃ or subsequent generation, wherein the F₃ or subsequent generation is produced by intercrossing, backcrossing, selfing, and/or producing double haploids from the F₂ generation and/or a subsequent generation; (b) genotyping one or more plants of a predicted population with respect to the plurality of markers, wherein each of the one or more plants of the predicted population is a descendant of two parents and each parent has at least 80% genetic identity to at least one of the two parental plants employed to generate the reference population; (c) summing the marker effects determined in step (a) for each of the one or more plants of the predicted population based on the genotyping of step (b); and (d) predicting a phenotype of the one or more plants of the predicted population based on the sum of the marker effects from step (c). In some embodiments, the reference population comprises a plurality of members of an F₃ or later generation generated by producing double haploids from the F₂ generation.

In some embodiments, the reference population is a reference network comprising a plurality of members generated by (i) selecting a plurality of different parental lines; (ii) crossing the plurality of different parental lines to produce a plurality of F₁ generations; (iii) intercrossing or backcrossing members of each F₁ generation to produce a plurality of distinct F₂ generations, and optionally singly or sequentially intercrossing, backcrossing, selfing, and/or producing double haploids from the plurality of distinct F₂ generations to produce distinct F₃ and, optionally, subsequent generations; (iv) pooling some or all of the members of the distinct F₂, F₃, or subsequent generations to generate the reference network, wherein each member of the reference network derives its genome from two of the different parental lines. In some embodiments, the reference network comprises plants derived from fewer than all possible crosses amongst the plurality of different parental lines. In some embodiments, the plant of the predicted population is an F₂ or subsequent generation of a cross between two members of the plurality of different parental lines that is not included in the reference network. In some embodiments, the reference network comprises plants derived from all possible crosses amongst the plurality of different parental lines. In some embodiments, the plant of the predicted population is an F₂ or subsequent generation of a cross between two parents, each of which is at least 80% genetically identical to one of the plurality of different parental lines that were employed to generate the reference network. In some embodiments, the reference population comprises at least 50 members, optionally at least 100 members, optionally at least 150 members, and further optionally at least 200 members. In some embodiments, each member of the reference population, each of the one or more plants of the predicted population, or both are inbred plants or double haploids.

In some embodiments of the presently disclosed methods, the determining step comprises estimating the marker effects for each of the plurality of markers by genome-wide best linear unbiased prediction (GBLUP). In some embodiments, the plurality of markers are sufficient to cover the genome of the plants of the reference population such that the average interval between adjacent markers on each chromosome is less than about 10 cM, optionally less than about 5 cM, optionally less than about 2 cM, and further optionally less than about 1 cM.

In some embodiments of the presently disclosed methods, the genotyping step comprising genotyping the one more plants as seeds, genotyping leaf tissue obtained from growing the one or more plants, or a combination thereof.

In some embodiments of the presently disclosed methods, predicting step (d) comprises employing a linear model for genome-wide best linear unbiased prediction (GBLUP) as set forth in Equation (4):

$${y}_{i} = μ + {\sum }_{j = 1}^{m} ( {z}_{ij} {g}_{j} ) + {e}_{i} , \qquad ( 4 )$$

wherein:
(i) yᵢ is the phenotypic BLUP of the line i, μ is the overall mean, zᵢⱼ is the genotype of the marker j for the line i, gⱼ is the effect of the marker j, and eᵢ the residual following eᵢ˜N(0, σₑ²);
(ii) μ is assumed to be a fixed effect and gⱼ is assumed to be a random effect following a normal distribution gⱼ˜N(0, σ_{gj}²);
(iii) each marker is assumed to have an equal genetic variance expressed by Equation (4a):

σ_{gj}²=σ_{g}²/m (4a),
with m the total number of markers used;
(iv) a variance-covariance matrix V for the phenotype y is expressed by Equation (4b):

$$V = {\sum }_{j = 1}^{m} ( {Z}_{j} {Z}_{j}^{T} {σ}_{gj}^{2} ) + {I}_{( n \times n )} {σ}_{e}^{2} \qquad ( 4 b )$$

wherein Zⱼ is a vector of genotypic scores of the marker j across n individuals in a population and I_{(n×n)} is an identity matrix with diagonal elements 1 and others 0;
(v) overall mean p, a fixed effect, is estimated as set forth in Equation (4c):

\hat{μ}=(X^{T}V⁻¹x)⁻¹X^{T}V⁻¹y (4c)
with X a vector of ones, and ĝⱼ, the effect of the marker j, is calculated as set forth in Equation (4d):

ĝⱼ=σ_{gj}²ZⱼV⁻¹(y−X\hat{μ}) (4d).

In some embodiments, the predicting step (d) is performed by a suitably-programmed computer

In some embodiments of the presently disclosed methods, the genetic identity between each parent and at least one of the two parental plants employed to generate the reference population is determined by calculating a percentage of shared pre-selected markers between each of the parents and the at least one of the two parental plants employed to generate the reference population.

In some embodiments, the presently disclosed methods further comprise isolating the leaf tissue from the one or more plants as the one or more plants are growing in a green house.

In some embodiments, the presently disclosed methods further comprise selecting one or more of the one or more plants of the predicted population that are predicted to have the phenotype of interest. In some embodiments, the selecting considers several traits of interest, and a multi-trait selection index is calculated for an individual in the predicted population. In some embodiments, the multi-trait selection index is calculated for a progeny individual in the predicted population using Equation (6):

$${I}_{i} = {\sum }_{j = 1}^{t} [ {w}_{j} \frac{{\hat{y}}_{i}^{j} - Min ( {\hat{y}}^{j} )}{Max ( {\hat{y}}^{j} ) - Min ( {\hat{y}}^{j} )} ] \qquad ( 6 )$$

and further wherein:
(i) Iᵢ is a multi-trait selection index for the progeny i;
(ii) wⱼ is a weight ranging from 0 to 1 for trait j used for measuring the relative importance of the trait j;
(iii) ŷᵢ^{j} is a predicted phenotype of the trait j (j=1, 2, . . . , t) in the progeny;
(iv) Min(ŷ^{j}) is a minimum value of the predicted phenotypes of the trait j in all the progeny in the predicted population; and
(v) Max(ŷ^{j}) is a maximum value of the predicted phenotypes of the trait j in all the progeny in the predicted population.
   In some embodiments, the multi-trait selection index calculation is performed by a suitably-programmed computer.

In some embodiments, the presently disclosed methods further comprise growing one or more of the one or more plants of the predicted population that are predicted to have the phenotype of interest in tissue culture or by planting.

The presently disclosed subject matter also provides methods for predicting phenotypes in plants of predicted populations by (a) determining marker effects for a plurality of markers in a genotyped and phenotyped reference population, wherein the reference population comprises a linkage disequilibrium (LD) panel; (b) genotyping one or more plants of the predicted population with respect to the plurality of markers, wherein each of the one or more plants of the predicted population is a descendant of two parents, each of which is at least 80% genetically identical to a member of the reference population; (c) summing the marker effects for each of the one or more plants of the predicted population based on the genotyping of step (b); and predicting the phenotype of the one or more plants of the predicted population based on the marker effects summed in step (c). In some embodiments, each of the one or more plant of the predicted population is an F₁ generation plant produced by crossing two members of the reference population or is an F₂ or subsequent generation plant produced by singly or multiply intercrossing, backcrossing, selfing, and/or producing double haploids from the F₁ generation plant or any subsequent generation thereof. In some embodiments, each of the plants of the predicted population is an F₁ generation plant produced by crossing two parental plants, each of which is at least 80% genetically identical to a member of the reference population. In some embodiments, the reference population comprises at least 50 members, optionally at least 100 members, optionally at least 150 members, optionally at least 200 members, and further optionally at least 250 members. In some embodiments, the determining step comprises calculating the marker effects for each of the plurality of markers by genome-wide best linear unbiased prediction (GBLUP). In some embodiments, the plurality of markers are sufficient to cover the genome of the plants of the reference population such that the average interval between adjacent markers on each chromosome is less than about 1 cM, optionally less than about 0.5 cM, and optionally less than about 0.1 cM. In some embodiments, each member of the reference population, each of the one or more plants of the predicted population, or both are inbred plants or double haploids.

In some embodiments, the presently disclosed methods further comprise identifying an core set of markers using a preselected significance level determined by a method of combining cross validations, single marker regression, and GBLUP and employing the core set of markers in summing step (c).

In some embodiments, the presently disclosed methods further comprise selecting one or more of the one or more plants of the predicted population that are predicted to have the phenotype of interest and reproducing the same in tissue culture or by planting.

The presently disclosed subject matter also provides methods for generating a plant with a phenotype of interest. In some embodiments, the methods comprise (a) determining marker effects for a plurality of markers in a genotyped and phenotyped reference population, wherein the reference population comprises (i) an F₂ generation produced by crossing two parental plants to produce an F₁ generation and then intercrossing, backcrossing, and/or selfing the F₁ generation; and/or (ii) an F₃ or subsequent generation, wherein the F₃ or subsequent generation is produced by intercrossing, backcrossing, selfing, and/or producing double haploids from the F₂ generation and/or a subsequent generation; and/or (iii) a reference network comprising a plurality of members generated by (1) selecting a plurality of different parental lines; (2) crossing the plurality of different parental lines to produce a plurality of F₁ generations; (3) intercrossing, backcrossing, and/or selfing the F₁ generation; and/or making a double haploid from F₁ to produce a plurality of distinct F₂ generations, and optionally singly or sequentially intercrossing, backcrossing, selfing, and/or producing double haploids from the plurality of distinct F₂ generations to produce distinct F₃ and, optionally, subsequent generations; (4) pooling some or all of the members of the distinct F₂, F₃, or subsequent generations to generate the reference network, wherein each member of the reference network derives its genome from two of the parental lines; and/or (5) a linkage disequilibrium (LD) panel; (b) genotyping one or more plants of a predicted population with respect to the plurality of markers, wherein the each of the one or more plants of the predicted population is a descendant of two parents each of which is at least 80% genetically identical to at least one of the two plants that comprise or where employed to generate the reference population; (c) summing the marker effects for each of the one or more plants of the predicted population based on the genotype determined in step (b) to generate a genetic score for each of the one or more plants of the predicted population; (d) predicting phenotypes of the one or more plants of the predicted population based on the genetic scores generated in step (c); (e) selecting one or more of the one or more plants of the predicted population based on the predicting step that are predicted to have a phenotype of interest, and (f) growing the selected one or more plants of the predicted population, wherein a plant with a phenotype of interest is generated. In some embodiments, the selecting step comprises selecting those plants of the predicted population that have a genetic score that exceeds a pre-selected threshold.

The presently disclosed subject matter also provides methods for estimating genetic similarity between a first and a second population. In some embodiments, the methods comprise (a) providing a first and a second population, wherein (i) the first population comprises individuals that are F₂ or subsequent generation progeny produced by crossing a first parent and a second parent to produce a first F₁ generation, and then intercrossing, backcrossing, selfing, and/or producing double haploids from the first F₁ generation to produce the F₂ generation, and optionally, further intercrossing, backcrossing, selfing, and/or producing double haploids from the F₂ generation and any subsequent generations to produce the first population; and (ii) the second population comprises individuals that are F₂ or subsequent generation progeny produced by crossing a third parent and a fourth parent to produce a second F₁ generation, and then intercrossing, backcrossing, selfing, and/or producing double haploids from the second F₁ generation to produce the F₂ generation, and optionally, further intercrossing, backcrossing, selfing, and/or producing double haploids from the F₂ generation and any subsequent generations to produce the second population; (b) genotyping the first, second, third, and fourth parents with respect to a plurality of pre-determined markers; (c) calculating first, second, third, and fourth percent genetic similarities, wherein (iii) the first percent genetic similarity is the percentage of allele sharing across all of the pre-determined markers of the first parent with respect to the third parent; (iv) the second percent genetic similarity is the percentage of allele sharing across all of the pre-determined markers of the first parent with respect to the fourth parent; (v) the third percent genetic similarity is the percentage of allele sharing across all of the pre-determined markers of the second parent with respect to the third parent; and (vi) the fourth percent genetic similarity is the percentage of allele sharing across all of the pre-determined markers of the second parent with respect to the fourth parent; (d) determining a first mean percentage genetic similarity comprising the mean percentage genetic similarity of the first percent genetic similarity and the third percent genetic similarity; (e) determining a second mean percentage genetic similarity comprising the mean percentage genetic similarity of the second percent genetic similarity and the fourth percent genetic similarity; and (f) selecting the greater of the first mean percentage genetic similarity and the second mean percentage genetic similarity, wherein the greater of the two mean percentage genetic similarities provides an estimate of the genetic similarity between a first and a second population. In some embodiments, the first population and the second population consist of F₄ progeny produced by selfing F₁, F₂, and F₃ individuals from the first F₁ population and the second F₁ population, respectively. In some embodiments, the plurality of pre-determined markers span substantially the entire genomes of the first and second populations.

Thus, it is an object of the presently disclosed subject matter to provide methods for predicting a phenotype in a plant in a predicted population.

An object of the presently disclosed subject matter having been stated hereinabove, and which is achieved in whole or in part by the presently disclosed subject matter, other objects will become evident as the description proceeds when taken in connection with the accompanying Figures as best described herein below.

### BRIEF DESCRIPTION OF THE FIGURES

FIG. 1 depicts a representative breeding scheme for an exemplary embodiment of the presently disclosed subject matter (PUP1).

FIG. 2 depicts a representative method for calculating genetic similarity between a predicted population and a candidate reference population in PUP1.

FIG. 3 is a bar graph showing a representative frequency distribution of accuracies of predictions using QTL-based prediction (gray bars) and PUP1 (black bars) when the genetic similarities between predicted and reference populations were greater than 0.80. QTL-based prediction was used to first identify significant QTL markers with the test statistic log of the odds (LOD) greater than an empirical LOD threshold estimated from 5000 permutations (Churchill & Doerge, 1994) using a procedure similar to composite interval mapping (CIM: Zeng, 1994), and then the effects of the markers were calculated by multiple regression in a reference population. PUP1 was used to calculate the effect of each marker in a genome using GBLUP (Meuwissen et al., 2001) without the identification of QTL in a reference population.

FIG. 4 depicts a representative breeding scheme for two additional exemplary embodiments of the presently disclosed subject matter (PUP2; Models 1 and 2).

FIG. 5 depicts a representative method for calculating genetic similarity between a predicted population and a network population in PUP2. In an exemplary embodiment of the method, the genetic similarities between A from a predicted population and each of four parents C, D, E, and G can be tested. In this example, parent D is identified as the one showing the closest genetic similarity to A. Genetic similarities between another parent B in the predicted population and the parents in the reference population other than D are determined since D has been identified as having the closest genetic similarity to A.

FIG. 6 depicts a representative breeding scheme for an exemplary embodiment of the presently disclosed subject matter (PUP3).

FIG. 7 is a graph describing accuracies of prediction using cross validation tests based on 100 replicates of cross validations performed at each significance level ranging from 1.0 to 1.00×10⁻⁶.

FIG. 8 is a scatter plot showing correlation relationships between PUP1-predicted and observed phenotypes of corn grain moisture.

FIG. 9 is a series of bar graphs showing the determined accuracies of predictions of a corn moisture phenotype using QTL-based prediction (gray bars) and PUP1-based prediction (black bars) in a corn breeding project as a representative example.

FIG. 10 is a scatter plot showing the relationships between genetic similarities among predicted and reference populations and the accuracies of predictions using PUP1 (open circles) vs. QTL-based predictions (filled circles). In this Figure, the shaded area to the right of 0.8 on the x-axis corresponds to data points with respect to predicted and reference populations that were at least 80% genetically identical.

FIG. 11 depicts a connection structure of a network population composed of 5 bi-parental subpopulations that share a common parent (A)

FIG. 12 is a scatter plot showing correlation relationships between PUP2-predicted and observed phenotypes of grain moisture.

FIG. 13 depicts a representative method that can be used for testing the accuracy of PUP2 based on real data analysis.

FIG. 14 is a series of bar graphs showing accuracies of predictions for an exemplary trait (corn moisture) using QTL-based predictions (gray bars) and PUP2-based predictions (black bars). The accuracies of the predictions for corn moisture employing QTL-based prediction and PUP2 using 78 bi-parental populations from 9 network populations are shown. In these initial studies, genetic similarity was not used in the selection of a reference network population for a given predicted population. QTL-based prediction was used to first identify significant QTL markers using a procedure similar to composite interval mapping (CIM: Zeng, 1994) using the model shown in Equation (7) below, and then the effects of the markers were calculated by multiple regression in a reference population.

FIG. 15 is a series of bar graphs showing the determined accuracies of predictions of a corn moisture phenotype using PUP1-based predictions (gray bars) and PUP2-based predictions (black bars) with Network 9 (see Table 12 below) as a representative reference population. The phenotypic and genotypic data used in PUP1 and PUP2 analysis were the same as those used to generate FIG. 3.

FIG. 16 is a scatter plot showing a relationship between genetic similarities among predicted and reference network populations and the accuracies of predictions using PUP2 (open circles). QTL-based predictions (filled circles) were used to first identify significant QTL markers using a procedure similar to composite interval mapping (CIM: Zeng, 1994) using the model shown in Equation (7) below, and then the effects of the markers were calculated by multiple regression in a reference population. PUP2 was used to calculate the effect of each marker on a genome using the model shown in Equation (7) without the identification of QTL in a reference population The shadowed region between 0.8 and 1 on the x-axis of FIG. 16 represents a focused area of PUP2 wherein the selected genetic similarity criterion was greater than 0.80.

FIG. 17 is a series of bar graphs of the frequency distribution of the accuracies of the predictions using QTL-based predictions (gray bars) and PUP2-based predictions (black bars) when the genetic similarities among predicted and reference populations were greater than 0.80 (in contrast to the data depicted in FIG. 9, in which genetic similarity was not considered). QTL-based prediction was used to first identify significant QTL markers using a procedure similar to composite interval mapping (CIM: Zeng, 1994) using the model shown in Equation (7), and then the effects of the markers were calculated by multiple regression in a reference population. PUP2 was used to calculate the effect of each marker on a genome using the model shown in Equation (7) without the identification of QTL in a reference network population.

### DETAILED DESCRIPTION

In general, observable traits are of two types: quantitative and qualitative. A quantitative trait such as corn yield or grain moisture shows continuous variation, while a qualitative trait such as corn disease resistance shows discrete variation. The expression of a trait is referred to as its “phenotype”. The phenotype of a qualitative trait is typically determined by one or a few major genes, while the phenotype of a quantitative trait is often determined by interactions among many genes, each with a small to moderate impact on the overall phenotype.

A locus on a chromosome that contributes to the phenotype of a quantitative trait is referred to as a “quantitative trait locus” (QTL). QTL mapping is a process for identifying statistical associations between phenotypes and the presence or absence of particular QTLs (i.e., collectively referred to as the “genotype”). For QTL mapping, this association can be modeled as set forth in Equation (1):

$${y}_{j} = μ + {\sum }_{i = 1}^{P} {G}_{i} {a}_{i} + {e}_{j} \qquad ( 1 )$$

where yⱼ is the phenotype of the progeny j in a given population, μ is the overall mean of the phenotype for the trait of interest, Gᵢ is the genotypic score of gene I which is translated from the genotype of the gene based on the coding rule described in Section II.A.2, aᵢ is the effect of gene i related to the phenotype of the trait which can be considered as the part of phenotype attributed to a gene, and eⱼ is the residual after the effects of all the genes are accounted for from the phenotype in the model, which, in general, is assumed to follow a normal distribution eⱼ˜N (0, σ²) with σ² being the environmental error. In the model, the phenotype yⱼ and the genotypic score Gᵢ are known quantities. In general, the phenotype yⱼ of the line j is the observable characteristic of a trait such as crop yield which is measured as the weight of all the seeds harvested from a plant in the field. In the model, genotype is defines as the genetic constitution of a plant. The genotypic score Gᵢ can be coded following the coding rule described in Section II.A.2. In the model, genotype is defined as If there are interactions (two-way interactions) between different genes, these interactions can be easily incorporated as covariates, simply products of the genotypic scores of any two genes, into the model.

A first step for QTL mapping is to identify and/or generate a mapping population. Suppose P₁ and P₂ are two inbred parents. Crossing P₁ and P₂ produces F₁ progeny (collectively referred to as the “F₁ generation”, or more simply, the “F₁”). Selfing one, some, or all of the F₁ generation results in F₂ progeny, and continued selfing of progeny for several generations results in an Fₙ generation (with n in some embodiments being equal to 3, 4, 5, 6, or more) and, if desired, the generation of recombinant inbred lines (RILs), each member of which is homozygous at every locus. These types of populations are also called bi-parental segregation populations due to genotypic segregation at one or more loci in the progeny of such populations, which renders them useful for QTL mapping.

A goal of QTL mapping is to identify those markers that show significant associations with the traits of interest. Such markers can be used to predict the breeding value of a line in a segregation population using Equation (2):

$$\hat{y} = μ + {\sum }_{i = 1}^{qtl\_num} {z}_{i} {a}_{i} \qquad ( 2 )$$

where ŷ is the estimated breeding value defined as the part of phenotype attributed to markers and zᵢ the genotypic score of the QTL I coded using the rule described in Section II.A.2. This is the fundamental model for marker-assisted breeding (MAS) in plant and animal breeding.

MAS is a procedure that includes two basic steps (Lande & Thompson, 1990). In the first step, QTL markers are identified by QTL mapping methods such as stepwise regression (Hocking, 1976). These markers are then added to a model and the effects of the markers are estimated by the regression of phenotypes on marker genotypes. In the second step, these estimated effects are used to predict the breeding value of a progeny in a population using Equation (2) above.

It was expected that MAS would reshape breeding programs and facilitate rapid gains from selection of superior progeny (Jannink et al. 2010). In comparison to conventional phenotypic selection methods, the primary advantages of MAS include: (i) short generation interval; (ii) more accurate selection based on QTLs and/or genes; and (iii) decreased costs of phenotyping. Simulation studies suggested that the short-term genetic gain from MAS was higher than that from purely phenotypic selection considering multi-cycle MAS performed per unit time (Hospital et al., 1997).

However, the actual gain due to MAS has been very limited for quantitative traits such as crop yield. A potential explanation for the low genetic gain is that it is difficult to identify all QTLs that are associated with some traits (e.g., polygenic traits including, but not limited to abiotic stress resistance (such as drought tolerance, yield, grain moisture, lodging rate etc.) and biotic stress resistance (such as pathogen resistance, insect resistance, iron deficiency chlorosis tolerance, aluminum tolerance etc.) when many small-effect QTLs segregate and no substantial, reliable effects can be identified (Jannink et al., 2010). Additionally, QTL effects are overestimated in many QTL studies (Beavis, 1998). This is because only QTL with large effects can be likely detected based on a given threshold for QTL identification, while those QTL with small effects cannot be identified.

Certain disadvantages of MAS can be minimized by genomic selection (Meuwissen et al., 2001). Genomic selection is a method of predicting breeding values by including genome-wide markers in a prediction system. Genomic selection has at least two primary advantages. First, it can reduce the risk of missing small-effect QTLs used for prediction (Rex & Yu, 2007). Second, it can provide more accurate estimates of QTL marker effects. Results from both simulation studies and real data validations have suggested that genomic prediction or selection might be a useful approach for generating improved individuals with respect to complex traits (Hayes et al., 2009).

Genomic selection has been applied to select progeny with advantageous genotypes within a bi-parental population in plant breeding (Rex & Yu, 2007; Jannink et al., 2010). With this approach, a reference population (for example, an F₄ population) is first generated. Phenotyping and genotyping are both required in the reference population in order to estimate the effects of each marker based on phenotypic and genotypic data gathered from the reference population. As disclosed herein, the breeding value of each progeny in successive generations can be predicted by these estimated effects, and selection can be made based on the breeding values.

A drawback of currently used genomic selection in plant breeding is that it requires phenotyping a reference population: typically an F₄ or double hybrid (DH) population (see e.g., Rex & Yu, 2007; Jannink et al., 2010). The primary reason for generating this reference population is to make a training population from which the effects of markers can be estimated. In the standard breeding scheme proposed in Rex & Yu, 2007, this type of population was termed cycle 0, and both phenotyping and genotyping efforts were required. As such, selection of individuals with desired phenotypes cannot be accomplished until the phenotyping itself is completed, which typically can only take place after a full growing season.

The presently disclosed subject matter, on the other hand, does not require that a full growing season passes before individuals with desired phenotypes are selected. Instead, the selection of individuals can begin as early as the seeds of a population of the individuals are produced because the genotypes of the seeds can be quickly obtained by extracting DNA from the seeds or from tissues of the seeds. A superior or improved individual (i.e., a progeny individual with a given phenotype of interest) cannot be selected unless and until phenotyping is completed, although the genotypes of the individuals of a progeny generation can be easily determined. As a result, the early use of genomic selection is significantly delayed. In addition, most phenotyping efforts are wasted once selection is done. Typically, only about 5% of all tested individuals are promoted to the next cycle of selection, while the vast majority of tested individuals are discarded.

Provided herein are general methods for predicting unobserved phenotype (PUP) in individuals using only genetic information. These general methods can increase the accuracy of phenotype prediction using genomic markers. With PUP, superior progeny individuals from a typical bi-parental plant breeding population can be identified directly based on marker genotypes with no need for phenotyping, thereby saving breeding time and costs. In some embodiments, a higher accuracy of prediction of phenotype-unknown progeny is expected due to the introduction of genetic similarity to allow selectively choosing a sufficiently genetically similar reference population upon which to base subsequent predictions. Exemplary results disclosed herein demonstrated that an accuracy of at least about 0.4 can be achieved based on a minimum genetic similarity criterion of 0.8 (i.e., 80% genetic similarity with respect to a plurality of markers of interest). The disclosed methods can be used in large scale bi-parental breeding projects based on consideration of a set of molecular markers that permit capture of linkage disequilibrium (LD) between QTLs and markers that segregate in the progeny populations. When high density markers are used for genomic prediction as shown in more detail hereinbelow (see e.g., the discussion of the exemplary PUP3 embodiment in Section II.C. below), the presently disclosed methods can also be employed to select an optimal subset of markers that can be used to provide enhanced predictions of unobserved phenotypes. As such, disclosed herein are details of implementations of the basic PUP strategies, including but not limited to PUP1, PUP2, and PUP3.

### I. DEFINITIONS

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. For example, the phrase “a marker” refers to one or more markers. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100. Similarly, the term “plurality” refers to “at least two”, and thus refers to, for example, 2, 3, 4, 5, 6, 7, 8, 9, 10, 15, 20, 25, 30, 35, 40, 45, 50, 75, 100, or more of that entity, including but not limited to whole number values between 1 and 100 or greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods. Accordingly, unless indicated to the contrary, the numerical parameters set forth in this specification and attached claims are approximations that can vary depending upon the desired properties sought to be obtained by the presently disclosed subject matter.

As used herein, the term “accuracy” as it relates to prediction is defined as the correlation coefficient between predicted and observed phenotypes of the members of a predicted population.

As used herein, the term “allele” refers to a variant or an alternative sequence form at a genetic locus. In diploids, single alleles are inherited by a progeny individual separately from each parent at each locus. The two alleles of a given locus present in a diploid organism occupy corresponding places on a pair of homologous chromosomes, although one of ordinary skill in the art understands that the alleles in any particular individual do not necessarily represent all of the alleles that are present in the species.

As used herein, the phrase “associated with” refers to a recognizable and/or assayable relationship between two entities. For example, the phrase “associated with a trait” refers to a locus, gene, allele, marker, phenotype, etc., or the expression thereof, the presence or absence of which can influence an extent, degree, and/or rate at which the trait is expressed in an individual or a plurality of individuals.

As used herein, the term “backcross”, and grammatical variants thereof, refers to a process in which a breeder crosses a progeny individual back to one of its parents: for example, a first generation F₁ with one of the parental genotypes of the F₁ individual. In some embodiments, a backcross is performed repeatedly, with a progeny individual of each successive backcross generation being itself backcrossed to the same parental genotype.

As used herein, the term “chromosome” is used in its art-recognized meaning of the self-replicating genetic structure in the cellular nucleus containing the cellular DNA and bearing in its nucleotide sequence the linear array of genes.

As used herein, the terms “cultivar” and “variety” refer to a group of similar plants that by structural or genetic features and/or performance can be distinguished from other varieties within the same species.

As used herein, the phrase “elite line” refers to any line that is substantially homozygous and has resulted from breeding and selection for superior agronomic performance.

As used herein, the term “gene” refers to a hereditary unit including a sequence of DNA that occupies a specific location on a chromosome and that contains the genetic instruction for a particular characteristic or trait in an organism.

As used herein, the phrase “genetic gain” refers to an amount of increase in performance that is achieved through artificial genetic improvement programs. In some embodiments, “genetic gain” refers to an increase in performance that is achieved after one generation has passed (see Allard, 1960).

As used herein, the phrase “genetic map” refers to the ordered list of loci usually relevant to position on a chromosome.

As used herein, the phrase “genetic marker” refers to a nucleic acid sequence (e.g., a polymorphic nucleic acid sequence) that has been identified as associated with a locus or allele of interest and that is indicative of the presence or absence of the locus or allele of interest in a cell or organism. Examples of genetic markers include, but are not limited to genes, DNA or RNA-derived sequences, promoters, any untranslated regions of a gene, microRNAs, siRNAs, QTLs, transgenes, mRNAs, ds RNAs, transcriptional profiles, and methylation patterns.

As used herein, the term “genotype” refers to the genetic makeup of an organism. Expression of a genotype can give rise to an organism's phenotype, i.e. an organism's physical traits. The term “phenotype” refers to any observable property of an organism, produced by the interaction of the genotype of the organism and the environment. A phenotype can encompass variable expressivity and penetrance of the phenotype. Exemplary phenotypes include but are not limited to a visible phenotype, a physiological phenotype, a susceptibility phenotype, a cellular phenotype, a molecular phenotype, and combinations thereof. The phenotype can be related to choline metabolism and/or choline deficiency-associated health effects. As such, a subject's genotype when compared to a reference genotype or the genotype of one or more other subjects can provide valuable information related to current or predictive phenotypes. As such, the term “genotype” refers to the genetic component of a phenotype of interest, a plurality of phenotypes of interest, or an entire cell or organism. Genotypes can be indirectly characterized using markers and/or directly characterized by nucleic acid sequencing.

As used herein, the phrase “determining the genotype” of an individual refers to determining at least a portion of the genetic makeup of an individual and particularly can refer to determining a genetic variability in the individual that can be used as an indicator or predictor of phenotype. The genotype determined can be in some embodiments the entire genomic sequence of an individual, but generally far less sequence information is usually considered. The genotype determined can be as minimal as the determination of a single base pair, as in determining one or more polymorphisms in the individual.

Further, determining a genotype can comprise determining one or more haplotypes. Still further, determining a genotype of an individual can comprise determining one or more polymorphisms exhibiting linkage disequilibrium to at least one polymorphism or haplotype having genotypic value. As used herein, the phrase “genotypic value” refers to an actual effect of a haplotype on the phenotype of a trait, and it can be actually considered as the contribution of a haplotype to a trait. In some embodiments, the genotype value can be calculated by regression of phenotype on haplotypes.

As used herein, “haplotype” refers to the collective characteristic or characteristics of a number of closely linked loci within a particular gene or group of genes, which can be inherited as a unit. For example, in some embodiments, a haplotype can comprise a group of closely related polymorphisms (e.g., single nucleotide polymorphisms; SNPs).

As used herein, “linkage disequilibrium” (LD) refers to a derived statistical measure of the strength of the association or co-occurrence of two distinct genetic markers. Various statistical methods can be used to summarize LD between two markers but in practice only two, termed D′ and r2, are widely used (see e.g., Delvin & Risch 1995; Jorde, 2000).

As such, the phrase “linkage disequilibrium” refers to a change from the expected relative frequency of gamete types in a population of many individuals in a single generation such that two or more loci act as genetically linked loci. If the frequency in a population of allele S is x, that of allele s is x′, or a part, progeny, or tissue culture thereof, B is y, and or a part, progeny, or tissue culture thereof, b is y′, then the expected frequency of genotype SB is xy, that of Sb is xy′, that of sB is x′y, and that of sb is x′y′, and any deviation from these frequencies is an example of disequilibrium.

In some embodiments, determining the genotype of an individual can comprise identifying at least one polymorphism of at least one gene and/or at one locus. In some embodiments, determining the genotype of an individual can comprise identifying at least one haplotype of at least one gene and/or at least one locus. In some embodiments, determining the genotype of an individual can comprise identifying at least one polymorphism unique to at least one haplotype of at least one gene and/or at least one locus.

As used herein, the term “heterozygous” refers to a genetic condition that exists in a cell or an organism when different alleles reside at corresponding loci on homologous chromosomes. As used herein, the term “homozygous” refers to a genetic condition existing when identical alleles reside at corresponding loci on homologous chromosomes. It is noted that both of these terms can refer to single nucleotide positions; multiple nucleotide positions, whether contiguous or not; and/or entire loci on homologous chromosomes.

As used herein, the term “hybrid” when used in the context of a plant refers to a seed and the plant the seed develops into that result from crossing at least two genetically different plant parents.

As used herein, the term “hybrid” when used in the context of nucleic acids, refers to a double-stranded nucleic acid molecule, or duplex, formed by hydrogen bonding between complementary nucleotide bases. The terms “hybridize” and “anneal” refer to the process by which single strands of nucleic acid sequences form double-helical segments through hydrogen bonding between complementary bases.

As used herein when used in the context of a plant, the terms “improved” and “superior”, and grammatical variants thereof, refer to a plant (or a part, progeny, or tissue culture thereof) that as a consequence of having (or lacking) a particular allele of interest expresses a phenotype of interest or expresses a phenotype of interest to a greater or lesser degree (as desired) relative to another plant (or a part, progeny, or tissue culture thereof) that lacks (or has) the particular allele of interest.

As used herein, the term “inbred” refers to a substantially homozygous individual or line. It is noted that the term can refer to individuals or lines that are substantially homozygous throughout their entire genomes or that are substantially homozygous with respect to subsequences of their genomes that are of particular interest.

As used herein, the phrase “immediately adjacent”, when used to describe a nucleic acid molecule that hybridizes to DNA containing a polymorphism, refers to a nucleic acid that hybridizes to a DNA sequence that directly abuts a sequence of interest (e.g., a polymorphic nucleotide base position). For example, a nucleic acid molecule can be used in a single base extension assay to analyze whether a polynucleotide base position is “immediately adjacent” to the polymorphism.

As used herein, the phrase “interrogation position” refers to a physical position on a solid support that can be queried to obtain genotyping data for one or more predetermined genomic polymorphisms.

As used herein, the terms “introgression”, “introgressed”, and “introgressing” refer to both a natural and artificial process whereby genomic regions of one individual are moved into the genome of another individual by crossing those individuals. Exemplary methods for introgressing a trait of interest include, but are not limited to breeding an individual that has the trait of interest to an individual that does not, and backcrossing an individual that has the trait of interest to a recurrent parent.

As used herein, the term “isolated” refers to a nucleotide sequence (e.g., a genetic marker) that is free of sequences that normally flank one or both sides of the nucleotide sequence in a plant genome. As such, the phrase “isolated and purified genetic marker” can be, for example, a recombinant DNA molecule, provided one of the nucleic acid sequences normally found flanking that recombinant DNA molecule in a naturally-occurring genome is removed or absent. Thus, isolated nucleic acids include, without limitation, a recombinant DNA that exists as a separate molecule (including, but not limited to genomic DNA fragments produced by the polymerase chain reaction (PCR) or restriction endonuclease treatment) with less than the full complement of its flanking sequences present, as well as a recombinant DNA that is incorporated into a vector, an autonomously replicating plasmid, or into the genomic DNA of a plant as part of a hybrid or fusion nucleic acid molecule.

As used herein, the term “linkage” refers to a phenomenon wherein alleles on the same chromosome tend to be transmitted together more often than expected by chance if their transmission were independent. Thus, two alleles on the same chromosome are said to be “linked” when they segregate from each other in the next generation in some embodiments less than 50% of the time, in some embodiments less than 25% of the time, in some embodiments less than 20% of the time, in some embodiments less than 15% of the time, in some embodiments less than 10% of the time, in some embodiments less than 9% of the time, in some embodiments less than 8% of the time, in some embodiments less than 7% of the time, in some embodiments less than 6% of the time, in some embodiments less than 5% of the time, in some embodiments less than 4% of the time, in some embodiments less than 3% of the time, in some embodiments less than 2% of the time, and in some embodiments less than 1% of the time.

As such, “linkage” typically implies and can also refer to physical proximity on a chromosome. Thus, two loci are linked if they are within in some embodiments 20 centiMorgans (cM), in some embodiments 15 cM, in some embodiments 12 cM, in some embodiments 10 cM, in some embodiments 9 cM, in some embodiments 8 cM, in some embodiments 7 cM, in some embodiments 6 cM, in some embodiments 5 cM, in some embodiments 4 cM, in some embodiments 3 cM, in some embodiments 2 cM, and in some embodiments 1 cM of each other. Similarly, a locus of the presently disclosed subject matter is linked to a marker (e.g., a genetic marker) if it is in some embodiments within 20, 15, 12, 10, 9, 8, 7, 6, 5, 4, 3, 2, or 1 cM of the marker.

As used herein, the phrase “linkage group” refers to all of the genes or genetic traits that are located on the same chromosome. Within the linkage group, those loci that are sufficiently close together can exhibit linkage in genetic crosses. Since the probability of a crossover occurring between two loci increases with the physical distance between the two loci on a chromosome, loci for which the locations are far removed from each other within a linkage group might not exhibit any detectable linkage in direct genetic tests. The term “linkage group” is mostly used to refer to genetic loci that exhibit linked behavior in genetic systems where chromosomal assignments have not yet been made. Thus, in the present context, the term “linkage group” is synonymous with the physical entity of a chromosome, although one of ordinary skill in the art will understand that a linkage group can also be defined as corresponding to a region of (i.e., less than the entirety) of a given chromosome.

As used herein, the term “locus” refers to a position on a chromosome of a species, and which can encompass in some embodiments a single nucleotide, in some embodiments several nucleotides, and in some embodiments more than several nucleotides in a particular genomic region. In some embodiments, the terms “locus” and “gene” are used interchangeably.

As used herein, the terms “marker” and “molecular marker” are used interchangeably to refer to an identifiable position on a chromosome the inheritance of which can be monitored and/or a reagent that is used in methods for visualizing differences in nucleic acid sequences present at such identifiable positions on chromosomes. Thus, in some embodiments a marker comprises a known or detectable nucleic acid sequence. Examples of markers include, but are not limited to genetic markers, protein composition, peptide levels, protein levels, oil composition, oil levels, carbohydrate composition, carbohydrate levels, fatty acid composition, fatty acid levels, amino acid composition, amino acid levels, biopolymers, starch composition, starch levels, fermentable starch, fermentation yield, fermentation efficiency, energy yield, secondary compounds, metabolites, morphological characteristics, and agronomic characteristics. Molecular markers include, but are not limited to restriction fragment length polymorphisms (RFLPs), random amplified polymorphic DNA (RAPD), amplified fragment length polymorphisms (AFLPs), single strand conformation polymorphism (SSCPs), single nucleotide polymorphisms (SNPs), insertion/deletion mutations (Indels), simple sequence repeats (SSRs), microsatellite repeats, sequence-characterized amplified regions (SCARs), cleaved amplified polymorphic sequence (CAPS) markers, and isozyme markers, microarray-based technologies, TAQMAN® markers, ILLUMINA® GOLDENGATE® Assay markers, nucleic acid sequences, or combinations of the markers described herein, which define a specific genetic and chromosomal location. The phrase a “molecular marker linked to a QTL” as defined herein can thus refer in some embodiments to SNPs, Indels, AFLP markers, or any other type of marker that can be used to identify the presence or absence of particular genomic sequences.

In some embodiments, a marker corresponds to an amplification product generated by amplifying a nucleic acid with one or more oligonucleotides, for example, by the polymerase chain reaction (PCR). As used herein, the phrase “corresponds to an amplification product” in the context of a marker refers to a marker that has a nucleotide sequence that is the same as or the reverse complement of (allowing for mutations introduced by the amplification reaction itself and/or naturally occurring and/or artificial alleleic differences) an amplification product that is generated by amplifying a nucleic acid with a particular set of oligonucleotides. In some embodiments, the amplifying is by PCR, and the oligonucleotides are PCR primers that are designed to hybridize to opposite strands of a genomic DNA molecule in order to amplify a genomic DNA sequence present between the sequences to which the PCR primers hybridize in the genomic DNA. The amplified fragment that results from one or more rounds of amplification using such an arrangement of primers is a double stranded nucleic acid, one strand of which has a nucleotide sequence that comprises, in 5′ to 3′ order, the sequence of one of the primers, the sequence of the genomic DNA located between the primers, and the reverse-complement of the second primer. Typically, the “forward” primer is assigned to be the primer that has the same sequence as a subsequence of the (arbitrarily assigned) “top” strand of a double-stranded nucleic acid to be amplified, such that the “top” strand of the amplified fragment includes a nucleotide sequence that is, in 5′ to 3′ direction, equal to the sequence of the forward primer—the sequence located between the forward and reverse primers of the top strand of the genomic fragment—the reverse-complement of the reverse primer. Accordingly, a marker that “corresponds to” an amplified fragment is a marker that has the same sequence of one of the strands of the amplified fragment.

As used herein, the phrase “marker assay” refers to a method for detecting a polymorphism at a particular locus using a particular method such as but not limited to measurement of at least one phenotype (e.g., seed color, oil content, or a visually detectable trait such as corn and soybean grain yield, plant height, flowering time, lodging rate, disease resistance, aluminum tolerance, iron deficiency chlorosis tolerance, and grain moisture); nucleic acid-based assays including, but not limited to restriction fragment length polymorphism (RFLP), single base extension, electrophoresis, sequence alignment, allelic specific oligonucleotide hybridization (ASO), random amplified polymorphic DNA (RAPD), microarray-based technologies, TAQMAN® Assays, ILLUMINA® GOLDENGATE® Assay analysis, nucleic acid sequencing technologies; peptide and/or polypeptide analyses; or any other technique that can be employed to detect a polymorphism in an organism at a locus of interest.

As used herein, the phrase “native trait” refers to any existing monogenic or polygenic trait in a certain individual's germplasm. When identified through the use of molecular marker(s), the information obtained can be used for the improvement of germplasm through selective breeding of predicted populations as disclosed herein.

As used herein, the phrases “nucleotide sequence identity” refers to the presence of identical nucleotides at corresponding positions of two polynucleotides. Polynucleotides have “identical” sequences if the sequence of nucleotides in the two polynucleotides is the same when aligned for maximum correspondence. Sequence comparison between two or more polynucleotides is generally performed by comparing portions of the two sequences over a comparison window to identify and compare local regions of sequence similarity, The comparison window is generally from about 20 to 200 contiguous nucleotides. The “percentage of sequence identity” for polynucleotides, such as 50, 55, 60, 65, 70, 75, 80, 85, 90, 95, 98, 99 or 100 percent sequence identity, can be determined by comparing two optimally aligned sequences over a comparison window, wherein the portion of the polynucleotide sequence in the comparison window can include additions or deletions (i.e., gaps) as compared to the reference sequence for optimal alignment of the two sequences.

The percentage can be calculated by any method generally applicable in the field of molecular biology. In some embodiments, the percentage is calculated by: (a) determining the number of positions at which the identical nucleic acid base occurs in both sequences to the number of matched positions; (b) dividing the number of matched positions by the total number of positions in the window of comparison; and (c) multiplying the result by 100 to determine the percentage of sequence identity. Optimal alignment of sequences for comparison can also be conducted by computerized implementations of known algorithms, or by visual inspection. Readily available sequence comparison and multiple sequence alignment algorithms are, respectively, the Basic Local Alignment Search Tool (BLAST; Altschul et al., 1990; Altschul et al., 1997) and ClustalW programs (Larkin et al., 2007), both available on the internet. Other suitable programs include, but are not limited to, GAP, BestFit, Plot Similarity, and FASTA, which are part of the Accelrys GCG® Wisconsin Package available from Accelrys, Inc. of San Diego, Calif., United States of America. In some embodiments, a percentage of sequence identity refers to sequence identity over the full length of one of the sequences being compared. In some embodiments, a calculation to determine a percentage of sequence identity does not include in the calculation any nucleotide positions in which either of the compared nucleic acids includes an “n” (i.e., where any nucleotide could be present at that position).

As used herein, the phrase “phenotypic marker” refers to a marker that can be used to discriminate between different phenotypes.

As used herein, the term “plant” refers to an entire plant, its organs (i.e., leaves, stems, roots, flowers etc.), seeds, plant cells, and progeny of the same. The term “plant cell” includes without limitation cells within seeds, suspension cultures, embryos, meristematic regions, callus tissue, leaves, shoots, gametophytes, sporophytes, pollen, and microspores. The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “polymorphism” refers to the presence of one or more variations of a nucleic acid sequence at a locus in a population of one or more individuals. The sequence variation can be a base or bases that are different, inserted, or deleted. Polymorphisms can be, for example, single nucleotide polymorphisms (SNPs), simple sequence repeats (SSRs), and Indels, which are insertions and deletions. Additionally, the variation can be in a transcriptional profile or a methylation pattern. The polymorphic sites of a nucleic acid sequence can be determined by comparing the nucleic acid sequences at one or more loci in two or more germplasm entries. As such, in some embodiments the term “polymorphism” refers to the occurrence of two or more genetically determined alternative variant sequences (i.e., alleles) in a population. A polymorphic marker is the locus at which divergence occurs. Exemplary markers have at least two (or in some embodiments more) alleles, each occurring at a frequency of greater than 1%. A polymorphic locus can be as small as one base pair (e.g., a single nucleotide polymorphism; SNP).

As used herein, the term “population” refers to a genetically heterogeneous collection of plants that in some embodiments share a common genetic derivation.

As used herein, the phrase “predicted population” refers to a population or plants for which a phenotype of interest is to be predicted based on the methods and compositions disclosed herein. In some embodiments, a predicted population is a population for which genotype information is available, but phenotype information with respect to a trait of interest is not available. As disclosed herein, the phenotype of one or more members of a predicted population (referred to herein as a “predicted plant”, “predicted individual”, and/or “plant in a predicted population) can be predicted based on genotype information alone in view of marker effects that have been derived from genotype and phenotype information available in a reference population.

As used herein, the phrase “reference population” refers to a population of individuals (e.g., plants) for which genotype and phenotype information is available with respect to a trait of interest. In some embodiments, the members of reference populations can be genotyped with respect to one or more genetic markers that are associated with a trait of interest. Observation of the genotyped members of the reference population with respect to phenotype of the trait of interest (referred to herein as “phenotyping”) facilitates the determination of the effects of the presence or absence of the one or more genetic markers that are associated with the trait of interest (referred to herein as “marker effects”). These marker effects can then be used to predict the phenotype of members of a predicted population based solely on the genotypes of the members of the predicted population with respect to the genetic markers as disclosed herein.

In some embodiments, a reference population is a network population. As used herein, the phrase “network population” refers to a population comprising a plurality of progeny individuals resulting from a plurality of bi-parental crosses, such that each member of the network population traces its ancestry to at least one of the individuals that were employed in at least one of the bi-parental crosses. In some embodiments, a network population is produced from n parents that are employed in bi-parental crosses, and each of the n parents are crossed to each of the other n parents other than themselves. As such, in some embodiments a network population comprises n (n−1) genetically distinct F₁ individuals, and/or progeny individuals derived therefrom by intercrossing, backcrossing, selfing, and/or the creation of double hybrids. Methods for establishing network populations are disclosed in more detail herein.

As used herein, the term “primer” refers to an oligonucleotide which is capable of annealing to a nucleic acid target (in some embodiments, annealing specifically to a nucleic acid target) allowing a DNA polymerase to attach, thereby serving as a point of initiation of DNA synthesis when placed under conditions in which synthesis of a primer extension product is induced (e.g., in the presence of nucleotides and an agent for polymerization such as DNA polymerase and at a suitable temperature and pH). In some embodiments, a plurality of primers are employed to amplify nucleic acids (e.g., using the polymerase chain reaction; PCR).

As used herein, the term “probe” refers to a nucleic acid (e.g., a single stranded nucleic acid or a strand of a double stranded or higher order nucleic acid, or a subsequence thereof) that can form a hydrogen-bonded duplex with a complementary sequence in a target nucleic acid sequence. Typically, a probe is of sufficient length to form a stable and sequence-specific duplex molecule with its complement, and as such can be employed in some embodiments to detect a sequence of interest present in a plurality of nucleic acids.

As used herein, the term “progeny” refers to any plant that results from a natural or assisted breeding of one or more plants. For example, progeny plants can be generated by crossing two plants (including, but not limited to crossing two unrelated plants, backcrossing a plant to a parental plant, intercrossing two plants, etc.), but can also be generated by selfing a plant, creating a double haploid, or other techniques that would be known to one of ordinary skill in the art. As such, a “progeny plant” can be any plant resulting as progeny from a vegetative or sexual reproduction from one or more parent plants or descendants thereof. For instance, a progeny plant can be obtained by cloning or selfing of a parent plant or by crossing two parental plants and include selfings as well as the F₁ or F₂ or still further generations. An F₁ is a first-generation progeny produced from parents at least one of which is used for the first time as donor of a trait, while progeny of second generation (F₂) or subsequent generations (F₃, F₄, and the like) are in some embodiments specimens produced from selfings (including, but not limited to double haploidization), intercrosses, backcrosses, or other crosses of F₁ individuals, F₂ individuals, and the like. An F₁ can thus be (and in some embodiments, is) a hybrid resulting from a cross between two true breeding parents (i.e., parents that are true-breeding are each homozygous for a trait of interest or an allele thereof, and in some embodiments, are inbred), while an F₂ can be (and in some embodiments, is) a progeny resulting from self-pollination of the F₁ hybrids.

As used herein, the phrase “quantitative trait locus” (QTL; quantitative trait loci—QTLs) refers to a genetic locus or loci that control to some degree a numerically representable trait that, in some embodiments, is continuously distributed. When a QTL can be indicated by multiple markers, the genetic distance between the end-point markers is indicative of the size of the QTL.

As used herein, the phrase “recombination” refers to an exchange of DNA fragments between two DNA molecules or chromatids of paired chromosomes (a “crossover”) over in a region of similar or identical nucleotide sequences. A “recombination event” is herein understood to refer to a meiotic crossover.

As used herein, the phrases “selected allele”, “desired allele”, and “allele of interest” are used interchangeably to refer to a nucleic acid sequence that includes a polymorphic allele associated with a desired trait. It is noted that a “selected allele”, “desired allele”, and/or “allele of interest” can be associated with either an increase in a desired trait or a decrease in a desired trait, depending on the nature of the phenotype sought to be generated in an introgressed plant.

As used herein, the phrase “significant QTL markers” refers to QTL markers that are characterized by a test statistic LOD that is greater than an empirical LOD threshold estimated from 5000 permutations (see Churchill & Doerge, 1994).

As used herein, the phrase “single nucleotide polymorphism”, or “SNP”, refers to a polymorphism that constitutes a single base pair difference between two nucleotide sequences. As used herein, the term “SNP” also refers to differences between two nucleotide sequences that result from simple alterations of one sequence in view of the other that occurs at a single site in the sequence. For example, the term “SNP” is intended to refer not just to sequences that differ in a single nucleotide as a result of a nucleic acid substitution in one versus the other, but is also intended to refer to sequences that differ in 1, 2, 3, or more nucleotides as a result of a deletion of 1, 2, 3, or more nucleotides at a single site in one of the sequences versus the other. It would be understood that in the case of two sequences that differ from each other only by virtue of a deletion of 1, 2, 3, or more nucleotides at a single site in one of the sequences versus the other, this same scenario can be considered an addition of 1, 2, 3, or more nucleotides at a single site in one of the sequences versus the other, depending on which of the two sequences is considered the reference sequence. Single site insertions and/or deletions are thus also considered to be encompassed by the term “SNP”.

As used herein, the phrase “stringent hybridization conditions” refers to conditions under which a polynucleotide hybridizes to its target subsequence, typically in a complex mixture of nucleic acids, but to essentially no other sequences. Stringent conditions are sequence-dependent and can be different under different circumstances.

Longer sequences typically hybridize specifically at higher temperatures. An extensive guide to the hybridization of nucleic acids is found in Tijssen, 1993. Generally, stringent conditions are selected to be about 5-10° C. lower than the thermal melting point (Tm) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Exemplary stringent conditions are those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides).

Stringent conditions can also be achieved with the addition of destabilizing agents such as formamide. Additional exemplary stringent hybridization conditions include 50% formamide, 5×SSC, and 1% SDS incubating at 42° C.; or SSC, 1% SDS, incubating at 65° C.; with one or more washes in 0.2×SSC and 0.1% SDS at 65° C. For PCR, a temperature of about 36° C. is typical for low stringency amplification, although annealing temperatures can vary between about 32° C. and 48° C. (or higher) depending on primer length. Additional guidelines for determining hybridization parameters are provided in numerous references (see e.g., Ausubel et al., 1999).

As used herein, the phrase “TAQMAN® Assay” refers to real-time sequence detection using PCR based on the TAQMAN® Assay sold by Applied Biosystems, Inc. of Foster City, Calif., United States of America. For an identified marker a TAQMAN® Assay can be developed for the application in the breeding program.

As used herein, the term “tester” refers to a line used in a testcross with one or more other lines wherein the tester and the line(s) tested are genetically dissimilar. A tester can be an isogenic line to the crossed line.

As used herein, the term “trait” refers to a phenotype of interest, a gene that contributes to a phenotype of interest, as well as a nucleic acid sequence associated with a gene that contributes to a phenotype of interest.

As used herein, the term “transgene” refers to a nucleic acid molecule introduced into an organism or its ancestors by some form of artificial transfer technique. The artificial transfer technique thus creates a “transgenic organism” or a “transgenic cell”. It is understood that the artificial transfer technique can occur in an ancestor organism (or a cell therein and/or that can develop into the ancestor organism) and yet any progeny individual that has the artificially transferred nucleic acid molecule or a fragment thereof is still considered transgenic even if one or more natural and/or assisted breedings result in the artificially transferred nucleic acid molecule being present in the progeny individual.

### II. EXEMPLARY METHODS FOR PREDICTING UNOBSERVED PHENOTYPES

The presently disclosed subject matter provides three general methods for predicting unobserved phenotypes: (i) predicting a phenotype-unknown population using a single reference population (referred to herein as “PUP1”); (ii) predicting a phenotype-unknown population using a network population comprising two or more subpopulations (referred to herein as “PUP2”); and (iii) predicting a phenotype-unknown population using a representative sample of related and/or unrelated germplasm (including, but not limited to a linkage disequilibrium panel as defined herein).

II.A. PUP1: Predicting Unobserved Phenotypes of Progeny from a Single Bi-parental Reference Population using Genome-wide Molecular Markers

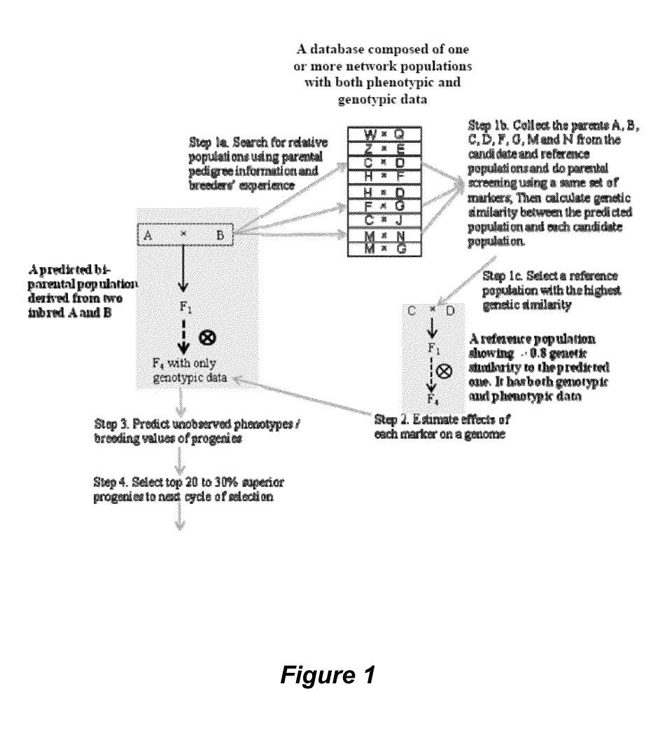

In some embodiments, the presently disclosed subject matter employ a single bi-parental reference population (referred to herein as “PUP1”). As shown in FIG. 1, PUP1 is a method for predicting the phenotypes for a trait of interest of individuals of a phenotype-unknown (i.e., predicted) population using a single bi-parental reference population for which both genotypic and phenotypic data with respect to the trait of interest is known or knowable (i.e., is known a priori or can be determined). In some embodiments, both genotypic and phenotypic data is known and/or knowable for the reference population, and only marker genotypic information is generated for the predicted population. The phenotypes of individuals in the predicted population are then predicted based on the genotypes determined for the individuals in the predicted population. In some embodiments, predicted populations result from new breeding projects while reference populations are previously generated populations for which genotypic and phenotypic information is already known (e.g., is stored in a database).

With respect to the genotypic information, the predicted and reference populations are in some embodiments genotyped using the same set of molecular markers based on a consensus genetic map. Under such circumstances, the genetic similarity between a predicted population and a reference population can be measured using these same markers (see Section II.A.1. hereinbelow). Another advantage is that it allows using the effects of QTL estimated from a reference population to predict the phenotypes of untested members of predicted populations using only genotypic data. This is a genetic basis for predicting phenotypes using PUP1.

In some embodiments of the presently disclosed subject matter, genome-wide markers are utilized for prediction, which differs significantly from conventional QTL-based prediction strategies. To highlight the advantages of the approach, the accuracies from both methods were compared and it was determined that the accuracy from PUP1 exceeded that from traditional QTL-based prediction by 27%. These results are illustrated and explained in more detail hereinbelow.

II.A.1. Choosing a Reference Population for a Predicted Population by Parental Molecular Marker Screening

For a given predicted population, several candidate reference populations can be selected based on criteria including, but not limited to pedigree information and breeding experience of breeders provided that both genotypic and phenotypic data are known or knowable (e.g., can be generated). The criteria used for the selection of a reference population can thus include: (i) high genetic similarity (e.g., genetic similarity including, but not limited to at least 0.70, 0.75, 0.80, 0.85, 0.90, 0.95, 0.97, 0.98, 0.99; i.e., all values greater than 0.70) with the predicted population; (ii) similar crop maturity to the predicted population; (iii) same tested locations; and/or (iv) a segregation of QTL in the population of interest (e.g., heritability on mean basis H²>0.40). These criteria can be employed to design a reference population that provides as much as QTL information similar to the predicted one.

Marker screening is conducted on the parents that generate the predicted and selected reference populations. In some embodiments, inbred individuals are employed as parents. In such embodiments, there is only one allele at each locus in each individual parental genome. Based on parental screening information, the genetic similarities between the reference populations and the predicted population can be calculated.

Choosing an appropriate reference population for PUP can thus enhance the accuracy of prediction. With respect to genetics, the accuracy can be affected by the genetic similarities between predicted and reference populations, which themselves can be calculated based on molecular markers using the methods disclosed herein. As used herein, the phrase “genetic similarity”, and grammatical variants thereof, refers to a degree to which the genomes of the individuals (i.e., the nucleotide sequence of the genomes) being compared are identical. It is recognized that genomes cannot typically be compared nucleotide-for-nucleotide on a genome-wide basis, and thus proxies for genome-wide comparisons can be employed in view of the fact that the actual nucleotide differences between members of the same species is likely to be very low.

In some embodiments, therefore, genetic similarity can be estimated by comparing the degree to which two or more individuals share relevant subsequences of their genomes. Such comparisons can include, but are not limited to determining to what extent two or more individuals share certain markers, which can include, but are also not limited to restriction fragment length polymorphisms (RFLPs), random amplified polymorphic DNA (RAPD), amplified fragment length polymorphisms (AFLPs), single strand conformation polymorphism (SSCPs), single nucleotide polymorphisms (SNPs), insertion/deletion mutations (Indels), simple sequence repeats (SSRs), microsatellite repeats, sequence-characterized amplified regions (SCARs), cleaved amplified and/or polymorphic sequence (CAPS) markers. In view of the fact that the methods of the presently disclosed subject matter relate in some embodiments to using genetic markers to predict unobserved phenotypes, genetic similarities can be estimated by determining what proportion of the genetic markers that are employed in the predictions are shared by the individuals being compared. Other methods for identifying, estimating, and/or calculating genetic similarity would be known to one of ordinary skill in the art, and include, but are not limited to calculations of genetic distances using the techniques of Nie (i.e., so-called “Nie's Distances”; see Nei & Roychoudhury, 1974; Nei, 1978; and references cited therein.

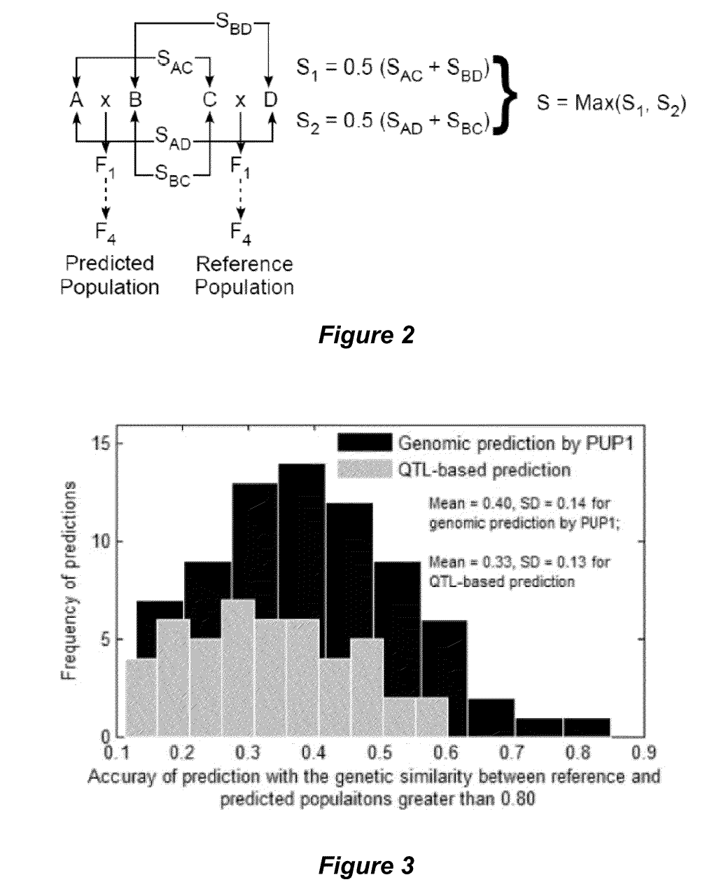

In some embodiments, genetic similarities are calculated using the exemplary method depicted in FIG. 2. With reference to FIG. 2, suppose that female A and male B are two inbred parents for a predicted population, and female C and male D are two parents for a reference population. The genetic similarity S_{AC} between females A and C (which is in some embodiments the proportion of allele sharing across all loci in a genome between A and C) can be calculated. The genetic similarity between males B and D can also be calculated as S_{BD}. The genetic similarity between the predicted and reference populations can be expressed as the average of S_{AC} and S_{BD} (i.e., S₁=0.5×(S_{AC}+S_{BD})). Similarly, the genetic similarity can be expressed as S₂=0.5×(S_{AD}+S_{BC}) based on a different combination of the females and males used to generate the two populations. In some embodiments, the genetic similarity between the populations is defined as the maximum genetic similarity between S₁ and S₂ (i.e., S=Max (S₁, S₂)).

In some embodiments, a population showing a sufficiently high genetic similarity (including, but not limited to at least 0.70, 0.75, 0.80, 0.85, 0.90, 0.95, 0.97, 0.98, 0.99; i.e., all values greater than 0.70) is chosen to be a reference population for a given predicted population. In some embodiments, a genetic similarity in excess of 0.80 can provide increased accuracy of prediction (measured in some embodiments as the correlation coefficient between predicted and observed phenotypes of progeny in a population) compared to QTL-based prediction (see FIG. 3). However, it is understood that the accuracy of prediction can vary with respect to different traits and/or genetic backgrounds of predicted and reference populations.

By way of example and not limitation, the prediction of corn moisture, one of the most important corn traits, was tested to define the relationship between genetic similarly and accuracy of prediction. As set forth in more detail hereinbelow in EXAMPLE 1, it was determined that a genetic similarity greater than 0.80 (i.e., 80% genetic similarity with respect to selected genetic markers) can be employed to obtain an accuracy of prediction which is greater than 0.40.

II.A.2. Estimating Effects of Each Marker from a Reference Population

In PUP1, a reference population is defined herein as a segregation population such as an Fₙ generation (wherein in some embodiments n=2, 3, 4, 5, or 6 and in some embodiments wherein the Fₙ generation is produced by iterative selfing of an F₁ individual), a recombinant inbred line (RIL), or a double haploid (DH) derived from two inbred parents. At least two types of data can be obtained from the reference population: (i) phenotypic data from a plurality (e.g., at least 25, 50, 100, 150, 200, 250, or more) of progeny for one or more traits of interest; and (ii) genotypic data of markers that in some embodiments are spread substantially throughout the genome. In some embodiments, the phenotypic data is from individuals grown under different growing conditions such as, but not limited to growing in multiple different locations (e.g., at least 2, 3, 4, 5, or more locations), which can provide better estimations of marker effects provided that sufficient phenotypic information is available.

Additionally, in some embodiments the markers are evenly distributed and/or of sufficient number to cover the entire genome or substantially the entire genome of the plants of the reference population. For example, the average interval between adjacent markers on each chromosome is in some embodiments less than 10 cM, in some embodiments less than 5 cM, in some embodiments less than 4 cM, in some embodiments less than 3 cM, in still another embodiment less than 2 cM, and in some embodiments less than 1 cM. The coverage information of the markers can be obtained by a genetic linkage map of the reference population. In some embodiments, most or all of the QTLs that are associated with the trait of interest are captured by the markers due to strong linkage disequilibrium between the QTLs and the markers.

By way of example and not limitation, genotypes of the markers used in the reference and predicted populations can be coded using the following exemplary rule: (i) if there are two different alleles α and β at a given locus, the genotype αα for a diploid plant with two alleles at each locus is coded as 0 and the genotype ββ is coded as 1. The heterozygous genotypes αβ and βα are coded as 0.5; (ii) if there are three alleles α, β, and γ at a given locus, the genotypes αα, ββ, and γγ are coded as 0, 1, and 2, respectively, and the heterozygous genotypes αβ, βγ, and αγ are coded as 0.5, 1.5, and 1, respectively. This exemplary coding rule is based only on additive effects of each allele. In some embodiments, dominant effects are excluded from the model since heterozygous genotypes make up a relatively minor proportion of most plant breeding populations employed.

Phenotypes from a reference population can be used to calculate genetic variance, which is a sum of genetic variations of all the QTL for the trait of interest, environmental variance which is caused by many environmental factors such as soil, temperature, water, fertilizer and so on, broad sense heritability (H²), which is a ratio of genetic variance over a sum of genetic variance and environmental variance; and best linear unbiased prediction (BLUPs) of each line across locations using the model of Equation (3):

yᵢⱼ=μ+Gᵢgᵢ+Lⱼbⱼ+eᵢⱼ (3)

where yᵢⱼ is the phenotype of the line i at the location j (which is an observable characteristic of a trait of interest); μ is the overall mean of the phenotype of a trait; Gᵢ is the indicator variable representing the genotype of the line i; gᵢ is the genotypic effect of the line i, which can be considered as a sum of QTL effects; Lⱼ is the indicator variable, with 1 indicating that the line has been phenotyped at the location j and 0 indicating that the line has not been phenotyped at the location; bⱼ is the effect of the location j caused by the difference of water, soil, temperature, and/or other factors; and eᵢⱼ is the residual of phenotype for the line i at the location j following eᵢⱼ˜N(0, σₑ²), Here, it is assumed that gᵢ is considered as a random effect following gᵢ˜N(0, σ_{g}²), and bⱼ is a fixed effect. The genetic variance σ_{g}² and environmental variance σₑ² can be estimated by restrained maximization likelihood estimation (REML; Henderson, 1975), and heritability is estimated as H²=σ_{g}²/(σ_{g}²+σₑ²/L) with L being the number of locations used for phenotyping. In the model, the parameter gᵢ can be calculated by a BLUP procedure developed by Henderson, 1975, and the BLUPs of each line are employed as phenotypes in the following model.

In some embodiments, the effect of each marker is estimated based on the phenotypic BLUPs and marker genotypic data from a reference population using genome-wide best linear unbiased prediction (GBLUP), BayesA, or BayesB (Meuwissen et al., 2001). In some embodiments of the presently disclosed subject matter, GBLUP was used for estimating marker effects. The linear model for GBLUP was:

$${y}_{i} = μ + {\sum }_{j = 1}^{m} ( {z}_{ij} {g}_{j} ) + {e}_{i} \qquad ( 4 )$$

where yᵢ is the phenotypic BLUP of the line i, μ is the overall mean, zᵢⱼ is the genotype of the marker j for the line i, gⱼ is the effect of the marker j, and eᵢ the residual following eᵢ˜N(0, σₑ²). In some embodiments, the phenotype BLUP can be the average of phenotypes of a line across multiple locations. Since a mixed model has been employed to calculate this quantity, it is called phenotype BLUP in the context of mixed model theory (Henderson 1975). In the model, μ is assumed to be a fixed effect and gⱼ is assumed to be a random effect following a normal distribution gⱼ˜N(0, σ_{gj}²). Each marker is also assumed to have an equal genetic variance expressed by Equation (4a):

σ_{gj}²=σ_{g}²/m (4a)

with m the total number of markers used (Meuwissen et al., 2001; Rex & Yu, 2007; Jannink et al., 2010). Based on the model, the variance-covariance matrix V for the phenotype y is expressed by Equation (4b):

$$V = {\sum }_{j = 1}^{m} ( {Z}_{j} {Z}_{j}^{T} {σ}_{gj}^{2} ) + {I}_{( n \times n )} {σ}_{e}^{2} \qquad ( 4 b )$$

where Zⱼ is a vector of genotypic scores of the marker j across n individuals in a population and I_{(n×n)} is an identity matrix with diagonal elements 1 and others 0. The overall mean μ, a fixed effect, can be estimated as set forth in Equation (4c):

\hat{μ}=(X^{T}V⁻¹X)⁻¹X^{T}V⁻¹y (4c)

with X a vector of ones, and the effect of the marker j can be calculated as set forth in Equation (4d):

ĝⱼ=σ_{gj}²ZⱼV⁻¹(y−X−\hat{μ}) (4d).

In some embodiments, one or more of Equations (4), (4a), (4b), (4c), and 4(d) are executed by a suitably-programmed computer.

II.A.3. Predicting Unobserved Phenotypes for a Predicted Population

Similar to the case with a reference population, a predicted population is defined as a segregation population such as an Fₙ generation (wherein in some embodiments n=2, 3, 4, 5, or 6, and in some embodiments wherein the Fₙ generation is produced by iterative selfing of F₁ and subsequent generation individuals), a recombinant inbred line (RIL), or a double haploid (DH) derived from two inbred parents. In general, it is not necessary to specify the number of predicted individuals and/or the number of markers used for the analysis. However, in some embodiments there are three general guidelines for making a predicted population: (i) the parents used for generating the population should be selected from lines with diverse traits of interest (including, but not limited to elite lines) and without killer traits such as severe susceptibility to plant disease; (ii) the number of progeny individuals in the predicted population should be sufficiently large (such as, but not limited to not less than 25, 50, 75, 100, or more) to ensure sufficient genetic variation for further selection; and (iii) the markers genotyped in the predicted population should be the same as those used to genotype the reference population to ensure straightforward projection of QTL and QTL by QTL interactions.

Based on the marker effects estimated as set forth herein, a phenotype for the trait of interest in a progeny in the predicted population can be calculated as set forth in Equation (5):

$${\hat{y}}_{i} = \hat{μ} + {\sum }_{j = 1}^{m} ( {z}_{ij} {\hat{g}}_{j} ) \qquad ( 5 )$$

where ĝⱼ is the effect estimated by Equation (4b) and zᵢⱼ is the genotype of the marker j of the line i. It can be seen that the phenotype of a progeny individual can be predicted by summing the effects of each marker present in the progeny individual. It can also be seen that this prediction model is an additive model which corresponds to the additive model used for estimating marker effects in the reference population. In some embodiments, the predicted population can be calculated as set forth in Equation (5) by a suitably-programmed computer.

II.A.4. Making a Selection in a Predicted Population

Selection of superior progeny individuals (i.e., progeny individuals predicted to express desirable phenotypes and/or have desirable genotypes with respect to one or more traits of interest) in a predicted population can be made based on its predicted phenotype for the trait of interest. By way of example and not limitation, the presently disclosed methods predict the phenotypes of individuals. After the predictions are made, seed from the individuals that are predicted to match the desired trait criteria are selected and only those seeds from individuals that meet these criteria (i.e., are of high predicted value) are grown for validation, thereby reducing or eliminating the need to validate “low-value” individuals.

To elaborate, two exemplary (i.e., non-limiting) strategies for selection are as follows: (i) select the top 30% of the progeny individuals based on total genetic score; and/or (2) discard the bottom 30% of the progeny individuals. The first strategy can be used for a trait with a high heritability (e.g., H²>0.5), and the second one can be used for a trait with a low heritability (e.g., H²<0.5). In practice, which strategy should be used can depend on breeding resources, genetic variation, goals of different breeding projects, and/or any other criteria of interest.

If several traits of interest are considered in selection, a multi-trait selection index can be calculated for a progeny individual in the predicted population using Equation (6):

$${I}_{i} = {\sum }_{j = 1}^{t} [ {w}_{j} \frac{{\hat{y}}_{i}^{j} - Min ( {\hat{y}}^{j} )}{Max ( {\hat{y}}^{j} ) - Min ( {\hat{y}}^{j} )} ] \qquad ( 6 )$$

where Iᵢ is the multi-trait selection index for progeny individual i, which is a weighted mean of genetic values of each trait for the progeny; wⱼ is the weight ranging from 0 to 1 for the trait j used for measuring the relative importance of the trait j; ŷᵢ^{j} is the predicted phenotype of the trait j (j=1, 2, . . . , t) in the progeny i using Equation (5); Min(ŷ^{j}) is the minimum value of the predicted phenotypes of the trait j in all the progeny in the predicted population; and Max(ŷ^{j}) the maximum value of the predicted phenotypes of the trait j in all the progeny in the predicted population. In some embodiments, the multi-trait selection index for a progeny individual is calculated by a suitably-programmed computer.

The multi-trait selection index is thus a weighted sum of the predicted phenotypes of each trait for a progeny. The weight used here is in some embodiments determined by breeders, representing the relative importance of a trait in a specific breeding project. For example, suppose there are three traits considered, and the weights for traits 1, 2, and 3 are 0.2, 0.3, and 0.5, respectively. Note the sum of these weights is equal to 1. These weights represent the relative importance of each trait from the perspective of breeding, and as such can be user-defined. In this case, trait 3 has 50% contribution in the overall multi trait index and can be ranked as the most important trait amongst the three traits.

II.B. PUP2: Predicting Unobserved Phenotypes in a Population from a Selected Reference Network Population using Genome-wide Molecular Markers

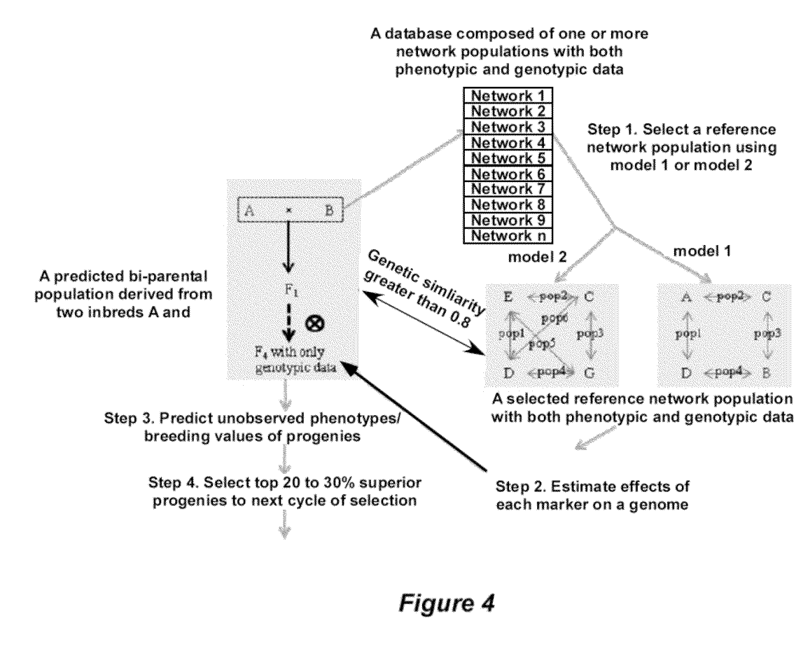

As an alternative to PUP1, in which the reference population was generated from a single bi-parental cross, PUP2 was developed to use a network population to improve prediction (see FIG. 4). A “network population” as defined herein is a set of bi-parental populations with shared and/or overlapping parents.

A parsimony method of assembling a network population using marker information is disclosed herein. In some embodiments, three steps are employed to prepare genetic data for the construction of a network: (i) parents are selected and used for a network; (ii) parents are genotyped using a set of molecular markers (parental screening); and (iii) pair-wise genetic similarity Sᵤ between the parents i and j is calculated using the method described in Section II.A.1.

By way of example and not limitation, a network population can be constructed as follows. In some embodiments, the generation of a network population starts by selecting a plurality of parents that as collectively display significant genetic divergence. As used herein, the phrase “significant genetic divergence” means that there is an overall genetic similarity among the plurality of parents of in some embodiments less than 0.70, in some embodiments less than 0.65, in some embodiments less than 0.60, in some embodiments less than 0.55, in some embodiments less than 0.50, in some embodiments less than 0.45, in some embodiments less than 0.40, in some embodiments less than 0.35, in some embodiments less than 0.30, in some embodiments less than 0.25, in some embodiments less than 0.20, in some embodiments less than 0.15, in some embodiments less than 0.10, and in some embodiments less than 0.05. Two of the plurality of inbred parents (arbitrarily designated as “P₁” and “P₂”) showing low genetic similarity (in some embodiments, those two inbred parents that are the least genetically identical from the plurality of inbred parents) are crossed. A third parent (arbitrarily designated as “P₃”) that shows low genetic similarity with P₁ and P₂ are then selected from the remaining parents and added into the network as a cross with P₁ or P₂. This process is then repeated until a desired number of crosses is reached (in some embodiments, all or nearly all of the crosses possible for the plurality of inbred parents, which in still further embodiments includes one, some, or all reciprocal crosses among the plurality of inbred parents).

A basic assumption of the PUP2 method described herein is that the genetic variation from all the populations within a network can be maximized by making crosses using parents that show long genetic distance (i.e., low genetic similarity). Another factor that can affect making a cross in plant breeding is the trait of interest. In general, breeders like to make a cross from two parents showing distinct phenotypes for the trait of interest. Thus, an exemplary method for constructing a network can combine marker and trait information from the parents.

In some embodiments, more alleles are introduced into a network reference population than in a simple bi-parental reference population. In PUP1, there are only two alleles in each reference population. One is from a female parent, and the other is from a male parent. When a network population is used, the number of alleles at a given locus can be increased by employing multiple parents with multiple (e.g., greater than 2) alleles at the given locus to generate the network population. This can ensure that enough alleles are present in the reference population to reflect all or substantially all of the alleles that exist in a given predicted population.

II.B.1. Selecting a Reference Network Population for a Given Predicted Population

For a given predicted population, a reference network population can be selected from a network population database defined as a collection of previously tested network populations for which both phenotypic and genotypic data are available or can be produced. In some embodiments, a same set of markers is used for genotyping the network and predicted populations.

Two basic embodiments have been developed based on the PUP2 approach and further based on different strategies for choosing a reference population. In Model 1, a reference network population is chosen (e.g., from a network population database) such that the two parents used to generate the predicted population are included in the reference network population. In Model 2, a reference network population is chosen such that the genetic similarities between the parents of the predicted population and two of the parents employed for generating the reference network population are both above a minimum cutoff (e.g., each parent used to generate the predicted population has a genetic similarity to one of the parents used to generate the reference network population of greater than 0.80). As such, Model 1 can be considered a special case of Model 2.

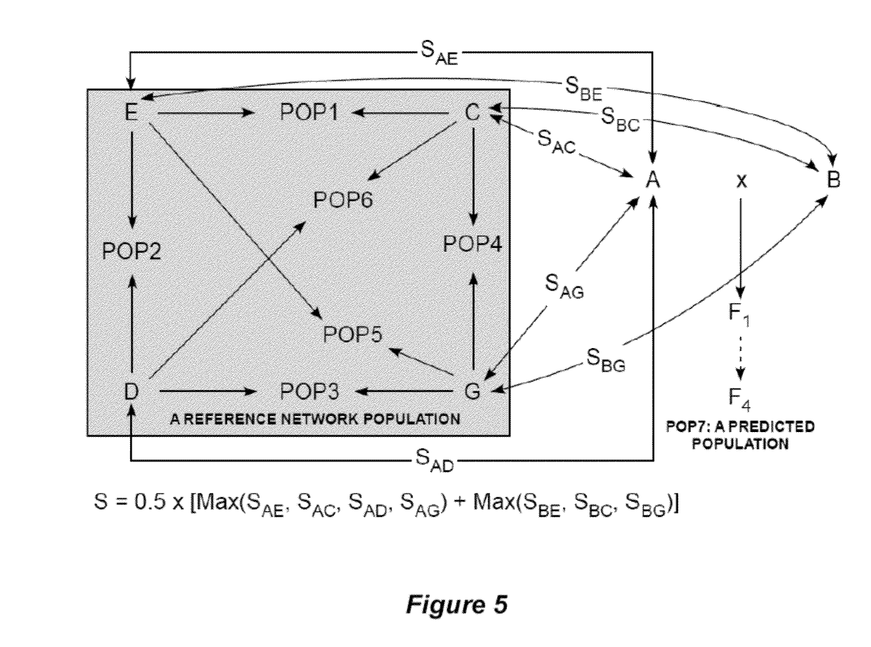

The genetic similarity used in Model 2 of PUP2 can in some embodiments be calculated based on parental marker screening data as exemplified in FIG. 5. As shown in the representative embodiment depicted in FIG. 5, suppose A and B are two inbred parents used to produce a predicted population, and C, D, E, and G are four parents used to produce a reference network population. Pairwise genetic similarities between one parent in the predicted population and one parent in the reference network population can be calculated, which in some embodiments is a proportion of shared alleles across all loci (in some embodiments, all assayed loci) in a genome. Then, a pair of parents showing the highest genetic similarity [Max (S_{AE}, S_{AG}, S_{AC}, S_{AD})] can be selected. After that, the other parent B of the predicted population can be compared with each of the parents other than the one to which parent A showed the highest genetic similarity (for example, D) in the network reference population, and Max (S_{BE}, S_{BG}, S_{BC}) can be used as a measure of genetic similarity between B and the remaining parents in the network. A reason for excluding D is that the genetic similarity between a predicted bi-parental population and a reference network population is defined as the one between four different parents where two parents are from the predicted population and the other two from the network population. D can thus be excluded so that the other parent that is closest in genetic similarity to B other than D from the remaining three parents in the network can be identified. Finally, the genetic similarity between the predicted and reference network populations can be measured as S=0.5×[Max (S_{AE}, S_{AG}, S_{AC}, S_{AD})+MaX (S_{BE}, S_{BG}, S_{BC})].

In some embodiments, the network population is selected to have one or more of the following properties: (i) close maturities for the subpopulations within a network; (ii) same locations for phenotyping; and (iii) a consensus linkage map combining marker data from different subpopulations. In some embodiments, the network population has each of the above properties simultaneously.

II.B.2. Estimating an Effect of Each Marker from a Reference Network Population

The effect of each marker can be estimated based on the phenotypic BLUPs and marker genotypic data from a reference population using genome-wide best linear unbiased prediction (GBLUP; Meuwissen et al., 2001). An exemplary linear model for GBLUP is:

$${y}_{ik} = μ + {x}_{k} {b}_{k} + {\sum }_{j = 1}^{m} ( {z}_{ikj} {g}_{j} ) + {e}_{ik} \qquad ( 7 )$$

where yᵢₖ is the phenotypic BLUP score of the progeny i in the population k, which is calculated by REML based on multiple location trait phenotypic data using model 3; μ is the overall mean of the phenotypes for all progenies; xₖ is an indicator variable with 1 representing the line comes from the population k and 0 representing the line does not come from the population k; bₖ is the effect of the of the population k, which is defined as the contribution of the population structure towards the phenotypic trait of interest; zᵢₖⱼ is the genotypic score of the marker j coded for the progeny i in the population k using the coding rule described hereinabove in Section II.A.1; gⱼ is the genetic effect of the marker j across all the populations; and eᵢₖ is the residual term after marker and population effects are accounted for in the model, which is assumed to follow eᵢₖ˜N(0, σₑ²). In the model, it is assumed that p and bₖ are fixed effects and gⱼ is a random effect following a normal distribution gᵢ˜N(0, σ_{gi}²). It is also assumed that each marker has an equal genetic variance σ_{gi}²=σ_{g}²/m, with m being the total number of markers.

II.B.3. Predicting Unobserved Phenotypes for a Predicted Population

Similar to PUP1, the phenotype of a progeny in a predicted breeding population can be predicted using Equation (5) hereinabove.

II.B.4. Making a Selection in a Predicted Population

Superior progeny with respect to single traits or multiple traits can be selected as set forth hereinabove with respect to the PUP1 method for further analysis such as, but not limited to field testing.

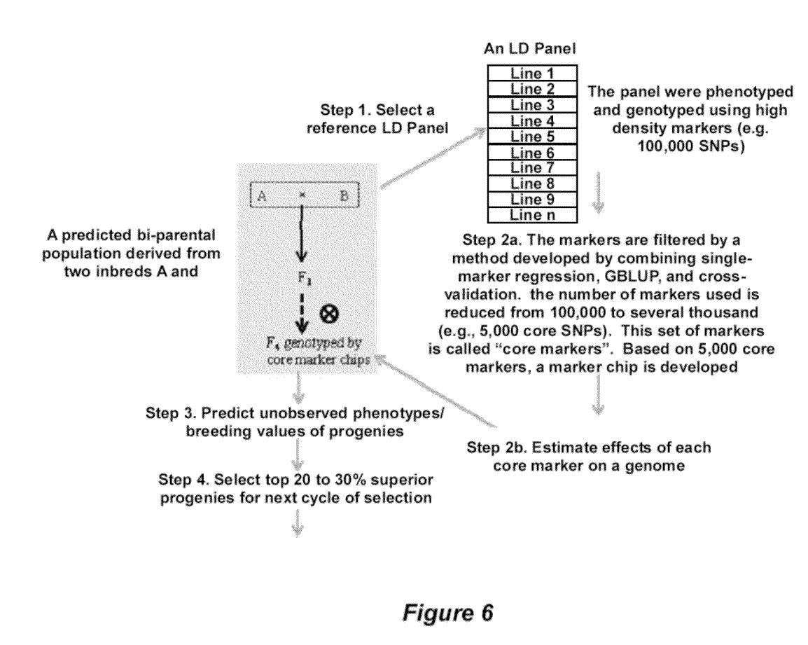

II.C. PUP3: Predicting Unobserved Phenotypes of Progeny in Populations from a Linkage Disequilibrium Panel including the Parents of the Predicted Population (see FIG. 6)

Although accuracy can be improved using PUP2 relative to QTL-based predictions or PUP1-based predictions, further improvement from the perspective of quantitative genetics and plant breeding can be gained using a third embodiment of the presently disclosed subject matter. Different from PUP1 and PUP2 based on traditional breeding populations, PUP3 employs a linkage disequilibrium (LD) panel as a reference population.

As used herein, the phrase “LD panel” refers to a collection of individual germplasm that includes a plurality of inbred germplasm. In some embodiments, the LD panel includes germplasm from at least 2, 3, 4, 5, 6, 7, 8, 9, 10, or more, including but not limited to at least 25, 50, 75, 100, or even several hundred inbred parents. Compared with PUP1 and PUP2 where particular crosses are needed to generate breeding populations, an LD panel can be assembled easily based upon germplasm stocks within a short time.

An exemplary LD panel harbors as much genetic diversity as possible, which can be beneficial in resolving complex trait variations of one or more genes (Yang et al., 2010). In PUP3, an LD panel is constructed in such a way that the lines included in the panel should explain greater than a pre-set minimum genetic variation of the germplasm (e.g., 70, 75, 80, 85, 90, 85, or more genetic variation). In some embodiments, PUP3 provides advantages over PUP2 since the allelic diversity present in an LD panel can often be higher than that present in the network populations employed in PUP2.

In some embodiments, high density markers are used to capture LD between QTL and markers. This is due to the LD decay caused by historical recombination. Compared to the several hundreds of markers typically used in PUP1 and PUP2 due to strong linkage disequilibrium of markers and QTLs in PUP1 and PUP2 populations, the number of markers employed in PUP3 can be very large since the linkage disequilibrium decays due to historical recombination among PUP3 lines and therefore more markers are needed to ensure to capture the linkage disequilibrium between QTL and makers. By way of example and not limitation, 10,000; 25,000, 50,000; 100,000; 250,000; 500,000; or even 1,000,000 SNP markers or more can be employed in the PUP3 embodiment (e.g., for corn and soybean gene discovery). With the development of second generation and other advanced DNA sequencing technologies, genotyping an individual with respect to more and more markers no longer limits the practical applications of LD analysis.

The ability to predict the phenotype of a line can be improved by using genomic prediction (Meuwissen et al., 2001; Meuwissen & Goddard, 2010). In genomic prediction, all assayable markers throughout the genome can be included in a model for predicting phenotypes of lines. Simulation studies showed a significant increase in genetic gain using genomic prediction as compared to MAS (Meuwissen et al., 2001; Yu and Rex 2007; Jannink et al, 2010), and results from cross-validation studies based on experimentally-derived data in animal and plant breeding further demonstrated and verified the merit of genomic prediction (Hayes et al., 2009).

However, studies to date have focused on the genotypic and phenotypic data from LD panels in animals, and a very complex effort in high density marker genotyping was required. PUP3, on the other hand, is a general method for combining an LD panel study with a large number of bi-parental breeding populations (e.g., F₄, RIL, and/or DH populations; see FIG. 6).

Viewed broadly, the generalized breeding scheme of PUP3 depicted in FIG. 6 includes four basic steps that are similar to the ones used in PUP1 and PUP2 but that differ in two respects. The first difference relates to a procedure for filtering genome-wide markers (in some embodiments, at least about 1,000,000 markers that can include, but are not limited to SNP markers) into a relative small subset of informative “core” markers (in some embodiments, about 5,000 informative core markers), wherein the subset of core markers provides an acceptable balance between the difficulty, time, and/or expense of assaying large numbers of genome-wide markers and the reduction in the level of prediction accuracy when fewer markers are employed. The second difference relates to the development of a chip that includes these core markers and that can be used to genotype some, most, or all relevant bi-parental populations using the chip. These two aspects of PUP3 are described in more detail herein, although it is understood that other aspects of PUP3 can be implemented using the corresponding strategies of PUP1 or PUP2 that are described hereinabove.

In some embodiments, not all markers (e.g., SNPs) or sequence information is employed in a model simultaneously. As discussed hereinabove, a gain from genomic prediction over conventional MAS can be obtained because all the QTLs associated with a trait of interest can be included in the model. However, this does not imply that when more markers are used, the accuracy of prediction is necessarily increased. In fact, including too many markers in a model can result in the introduction of increased noise into the model, especially when the GBLUP method is employed (see Meuwissen & Goddard, 2010). In order to find a proper balance between increased coverage and increased noise, a marker filter procedure (i.e., a strategy for using a subset of all available markers as a proxy rather than using all of the available markers per se) can be used.

In some embodiments, a simple method is used to filter markers from a starting population of all possible markers (in some embodiments, a genome-wide marker set can include 100,000; 500,000; 1,000,000; 2,000,000; 3,000,000; or more markers depending, for example, on genome size and the average genetic interval between markers that is desired) down to an informative subset of core markers (in some embodiments, a subset that includes several hundred to several thousand core markers).

For example, a single marker regression method where at statistic is obtained for a marker by the regression of phenotypes on genotypes can be employed (Liu, 1998). In some embodiments, the method includes the t test, ANOVA, or simple regression. The t test and ANOVA focus on testing the difference between phenotypic means of marker genotype classes, while simple regression provides an estimate of marker effect. At a marker, all of the predicted individuals can be split into distinct groups according to marker genotype and the phenotypic means of the groups are compared. In some embodiments, markers with p values greater than a predetermined significance level (including but not limited to 0.001, 0.005, 0.01, or 0.05) can be employed. As might be expected, the number of markers selected can vary with the significance level selected. However, there is generally no way to know a priori what particular significance level would provide the best (i.e., most accurate) prediction.

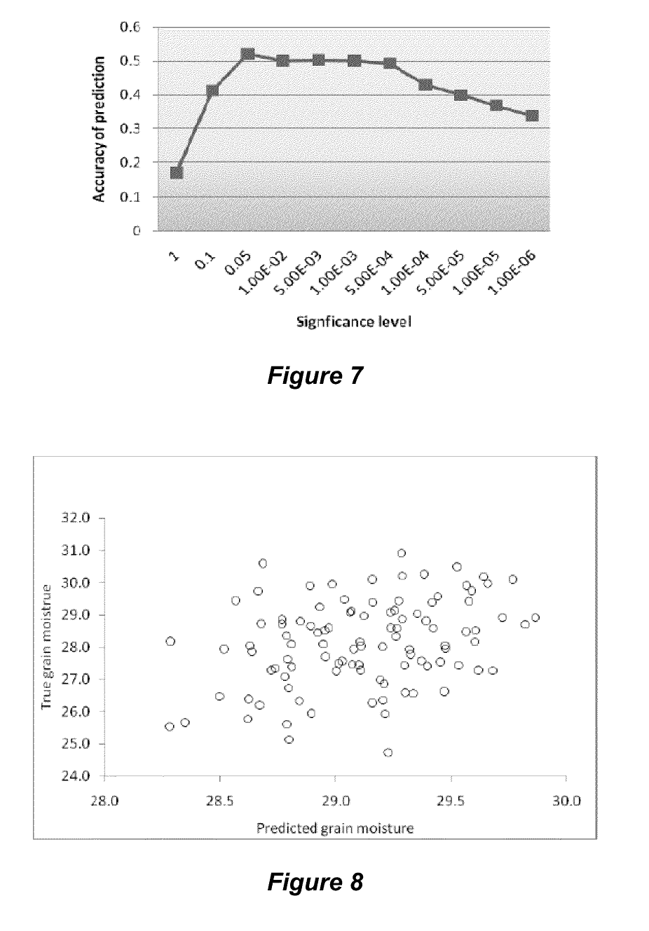

Thus, an approach to addressing this problem is disclosed herein. By way of example and not limitation, a set of sequential significance levels (e.g., a=1.0, 0.50, 0.30, 0.20, 0.10, 0.05, 0.01, 0.005, 0.001, 0.0005, 0.0001. etc.) can be created as exemplified in FIG. 7. When a=1.00, all possible markers are used. The most stringent significance level (i.e., the level at which no false positives are generated) is determined when there is no significant marker identified at that level. In some embodiments, QTL identification is stopped at this point. For a given a level—for example, when a=0.05−QTL markers are identified using single marker regression based on the t tests for individual associations between phenotype and marker genotype scores. The markers showing p values from t tests less than a=0.05 are identified as QTLs.

In the following, a whole sample is defined as a set of all lines with phenotypes and genotypic data of markers identified by single marker regression. Within each replicate, the whole sample is split randomly into two subsamples: a training sample made up of a fraction of the lines (e.g., 60% of the lines in the whole sample) and a validation sample made up of the remaining fraction of the lines (e.g., the remaining 40%). The effects of markers can be estimated using GBLUP as described in Section II.A.2. for a training dataset, which are then used to predict the phenotype of a line in a validation sample as described in Section II.A.3. The accuracy of prediction can be expressed as the correlation coefficient between the predicted and true phenotypes in the validation sample. The resulting accuracy is the average of the predictive accuracies over all of the replicates performed, and is recorded for the significance level used for QTL identification using single marker regression. This process is then repeated for all sequential significance levels and all of the accuracies obtained for each level are recorded. After that, a curve of accuracies vs. significance levels can be plotted, and in some embodiments the significance level corresponding to the highest accuracy can be selected as an appropriate level used for prediction (see FIG. 7 for a representative example).

For example and with reference to the curve depicted in FIG. 7, a=0.05 corresponding to 3000 SNPs in this example can be employed as a selected level to move forward, or a=5×10⁻⁴ corresponding to 1000 SNPs can be employed as a selected level to move forward in the example. Thereafter, all the significant markers are identified using single marker regression at the selected level, and only those markers are employed as a core marker set for future prediction. In practice, a marker chip can be constructed based on the core marker set. The effects of these markers are estimated using the GBLUP approach described in more detail hereinabove. These effects can then be used for genomic prediction in bi-parental breeding populations.

A next aspect of PUP3 is to genotype breeding populations using a chip that includes the core markers identified as described hereinbelow. It is expected that the number of core markers included in a chip would typically be at least about 1000 and in some embodiments as many as 5000 or more. Compared to chips with 50,000 or more SNPs, the core marker set chips can save genotyping costs. Additionally, they can reduce the time necessary for data analysis by removing from the chips (or, in some embodiments, not including on the chips) those markers that have no identifiable association with the trait of interest. As such, the phenotype of a progeny in a predicted population can be predicted based on genotypic data derived from the use of such core marker chips.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Exemplary PUP1 Implementation

The PUP1 method was employed to predict phenotypes in a predicted population based on marker genotypic data only. The reference population used was a F₄ population derived from two parents A and B, while the tested population was also a F₄ population derived from two parents A and C. Each F₄ population was produced by crossing the initial parents to produce an F₁, selfing the F₁ to produce an F₂, selfing the F₂ to produce an F₃, and selfing the F₃ to produce the F₄ populations. Both F₄ populations had parent A in common, so the genetic similarity between the two populations was determined by examining the different parents B and C. It was found that the genetic similarity between the reference and predicted populations was 0.78.

First, the effects of a series of markers present at loci throughout all ten Zea mays chromosomes were estimated in the reference population with respect to grain moisture. The positions of the markers and the estimated marker effects are presented in Table 1.

TABLE 1
Marker Effects Estimated in a Reference Population

| | Marker | Marker | Estimated |
|---|---|---|---|
| Chromosome | Name | Position (cM) | Marker Effect |
| 1 | SM0095C | 6.9 | 0.03 |
| 1 | SM0208B | 47.5 | −0.03 |
| 1 | SM1099B | 49.3 | −0.01 |
| 1 | SM0687C | 60.2 | 0.04 |
| 2 | SM0372B | 31.6 | −0.07 |
| 2 | SM0064A | 52.2 | −0.02 |
| 2 | SM0070C | 54.4 | −0.05 |
| 2 | SM0616A | 63.3 | −0.05 |
| 2 | SM0040B | 66.3 | −0.07 |
| 2 | SM0516A | 67.7 | −0.06 |
| 2 | SM0410D | 89.7 | −0.04 |
| 2 | SM0370A | 90.2 | 0.01 |
| 2 | SM1095A | 91.8 | 0.01 |
| 2 | SM0289B | 96.4 | −0.01 |
| 2 | SM1100A | 98.6 | 0.08 |
| 2 | SM0588B | 109.0 | 0.07 |
| 2 | SM0357A | 126.2 | 0.04 |
| 3 | SM0646D | 51.0 | −0.09 |
| 3 | SM0314B | 93.2 | 0.04 |
| 3 | SM0967A | 101.4 | 0.04 |
| 3 | SM0005B | 106.7 | 0.07 |
| 3 | SM0364B | 113.1 | 0.06 |
| 3 | SM0668H | 114.5 | 0.01 |
| 3 | SM0543A | 121.3 | −0.08 |
| 4 | SM0236A | 48.5 | −0.11 |
| 4 | SM0239A | 65.3 | 0.04 |
| 4 | SM0274A | 72.9 | −0.04 |
| 4 | SM0425A | 100.2 | −0.02 |
| 4 | SM0258B | 102.0 | −0.03 |
| 5 | SM0269B | 27.1 | 0.05 |
| 5 | SM0493B | 73.8 | −0.03 |
| 5 | SM0105C | 74.0 | 0.02 |
| 5 | SM0648A | 80.1 | 0.01 |
| 5 | SM0108C | 82.5 | −0.01 |
| 5 | SM0632H | 86.3 | 0.05 |
| 5 | SM0205B | 91.7 | 0.02 |
| 5 | SM0803D | 96.8 | −0.07 |
| 5 | SM0987C | 105.0 | −0.01 |
| 6 | SM0156B | 37.2 | −0.02 |
| 6 | SM0940E | 85.6 | −0.02 |
| 6 | SM0939C | 88.2 | 0.01 |
| 7 | SM0368A | 0.0 | −0.01 |
| 7 | SM0359F | 28.1 | −0.03 |
| 7 | SM0093B | 38.5 | −0.03 |
| 7 | SM0014F | 39.5 | −0.07 |
| 7 | SM0912D | 63.8 | 0.01 |
| 7 | SM0167B | 64.6 | −0.04 |
| 7 | SM0074D | 82.8 | 0.04 |
| 7 | SM0139B | 101.3 | 0.02 |
| 7 | SM0128E | 103.9 | −0.02 |
| 8 | SM0246B | 0.0 | −0.03 |
| 8 | SM0300B | 0.8 | −0.02 |
| 8 | SM0727B | 7.1 | 0.02 |
| 8 | SM1080D | 15.3 | 0.03 |
| 8 | SM0712B | 16.7 | −0.02 |
| 8 | SM0826B | 19.1 | −0.01 |
| 8 | SM0248D | 28.3 | 0.07 |
| 8 | SM0036B | 43.0 | 0.10 |
| 8 | SM0271A | 65.5 | −0.02 |
| 8 | SM0464D | 66.2 | 0.05 |
| 8 | SM0538A | 99.3 | 0.04 |
| 8 | SM0596E | 105.9 | −0.07 |
| 8 | SM0528B | 107.6 | −0.09 |
| 8 | SM0780C | 110.0 | 0.01 |
| 9 | SM0847C | 23.6 | −0.01 |
| 9 | SM0469A | 25.9 | −0.01 |
| 10 | SM0913B | 16.7 | 0.02 |
| 10 | SM0804F | 19.7 | 0.06 |
| 10 | SM0474B | 25.0 | 0.02 |
| 10 | SM1019B | 56.0 | −0.08 |
| 10 | SM0478A | 58.5 | −0.11 |
| 10 | SM0954B | 76.9 | −0.06 |
| 10 | SM0953C | 77.8 | 0.00 |
| 10 | SM0898A | 78.6 | −0.07 |

In the reference population, there were 45 individuals, and these individuals were phenotyped across five different growing locations. Each individual was genotyped using the above SNP markers, and the calculated effect of each SNP is listed in Table 1. These estimates were calculated using Equations (4), (4a), (4b), (4c), and (4d).

Next, the phenotypes with respect to corn grain moisture of the individuals in the predicted population were determined based on the marker genotypic data using Equation (5). The predicted population included 102 individuals, each of which was genotyped using 108 SNP markers. Among these markers, there were 27 markers that showed no segregation in the reference population, and thus no estimation for these marker effects was generated (see Table 2). The phenotype of each individual in the predicted population was calculated based on the remaining markers the effects of which were estimated in the reference population. Table 3 summarizes the predicted grain moisture for 102 individuals in the predicted population.

TABLE 2
Marker Information in a Predicted Population.

| | | Marker Position | Estimated |
|---|---|---|---|
| Chromosome | Marker name | (cM) | Marker Effect |
| 1 | SM0095C | 6.9 | 0.03 |
| 1 | SM0208B | 47.5 | −0.03 |
| 1 | SM1099B | 49.3 | −0.01 |
| 1 | SM0687C | 60.2 | 0.04 |
| 2 | SM0372B | 31.6 | −0.07 |
| 2 | SM0064A | 52.2 | −0.02 |
| 2 | SM0070C | 54.4 | −0.05 |
| 2 | SM0616A | 63.3 | −0.05 |
| 2 | SM0040B | 66.3 | −0.07 |
| 2 | SM0516A | 67.7 | −0.06 |
| 2 | SM0410D | 89.7 | −0.04 |
| 2 | SM0370A | 90.2 | 0.01 |
| 2 | SM1095A | 91.8 | 0.01 |
| 2 | SM0289B | 96.4 | −0.01 |
| 2 | SM1100A | 98.6 | 0.08 |
| 2 | SM0588B | 109.0 | 0.07 |
| 2 | SM0357A | 126.2 | 0.04 |
| 3 | SM0646D | 51.0 | −0.09 |
| 3 | SM0314B | 93.2 | 0.04 |
| 3 | SM0967A | 101.4 | 0.04 |
| 3 | SM0005B | 106.7 | 0.07 |
| 3 | SM0364B | 113.1 | 0.06 |
| 3 | SM0668H | 114.5 | 0.01 |
| 3 | SM0543A | 121.3 | −0.08 |
| 4 | SM0236A | 48.5 | −0.11 |
| 4 | SM0239A | 65.3 | 0.04 |
| 4 | SM0274A | 72.9 | −0.04 |
| 4 | SM0425A | 100.2 | −0.02 |
| 4 | SM0258B | 102.0 | −0.03 |
| 5 | SM0269B | 27.1 | 0.05 |
| 5 | SM0493B | 73.8 | −0.03 |
| 5 | SM0105C | 74.0 | 0.02 |
| 5 | SM0648A | 80.1 | 0.01 |
| 5 | SM0108C | 82.5 | −0.01 |
| 5 | SM0632H | 86.3 | 0.05 |
| 5 | SM0205B | 91.7 | 0.02 |
| 5 | SM0803D | 96.8 | −0.07 |
| 5 | SM0987C | 105.0 | −0.01 |
| 6 | SM0156B | 37.2 | −0.02 |
| 6 | SM0940E | 85.6 | −0.02 |
| 6 | SM0939C | 88.2 | 0.01 |
| 7 | SM0368A | — | −0.01 |
| 7 | SM0359F | 28.1 | −0.03 |
| 7 | SM0093B | 38.5 | −0.03 |
| 7 | SM0014F | 39.5 | −0.07 |
| 7 | SM0077A | 43.7 | −0.05 |
| 7 | SM0912D | 63.8 | 0.01 |
| 7 | SM0074D | 82.8 | 0.04 |
| 7 | SM0139B | 101.3 | 0.02 |
| 7 | SM0128E | 103.9 | −0.02 |
| 8 | SM0246B | — | −0.03 |
| 8 | SM0300B | 0.8 | −0.02 |
| 8 | SM0727B | 7.1 | 0.02 |
| 8 | SM1080D | 15.3 | 0.03 |
| 8 | SM0712B | 16.7 | −0.02 |
| 8 | SM0826B | 19.1 | −0.01 |
| 8 | SM0248D | 28.3 | 0.07 |
| 8 | SM0036B | 43.0 | 0.10 |
| 8 | SM0271A | 65.5 | −0.02 |
| 8 | SM0464D | 66.2 | 0.05 |
| 8 | SM0538A | 99.3 | 0.04 |
| 8 | SM0596E | 105.9 | −0.07 |
| 8 | SM0528B | 107.6 | −0.09 |
| 8 | SM0780C | 110.0 | 0.01 |
| 9 | SM0847C | 23.6 | −0.01 |
| 9 | SM0469A | 25.9 | −0.01 |
| 10 | SM0913B | 16.7 | 0.02 |
| 10 | SM0804F | 19.7 | 0.06 |
| 10 | SM0474B | 25.0 | 0.02 |
| 10 | SM1019B | 56.0 | −0.08 |
| 10 | SM0478A | 58.5 | −0.11 |
| 10 | SM0954B | 76.9 | −0.06 |
| 10 | SM0898A | 78.6 | −0.07 |

“—” indicates that the markers showed no segregation in the reference population, and therefore no estimation for the marker effect was possible.

To evaluate the accuracy of prediction using PUP1, grain moisture data were collected across the same locations as employed for the reference population (see Table 3). The accuracy of prediction was expressed as the correlation coefficient between the predicted and observed phenotypes. The accuracy of prediction was R=0.33 (see FIG. 8).

TABLE 3
Predicted and Measured Grain Moisture in a Predicted Population

| | Predicted | Observed |
|---|---|---|
| Individual | Grain | Grain |
| No. | Moisture | Moisture |
| 1 | 29.5 | 27.4 |
| 2 | 28.3 | 25.5 |
| 3 | 28.9 | 25.9 |
| 4 | 28.3 | 25.7 |
| 5 | 29.0 | 27.3 |
| 6 | 29.4 | 29.6 |
| 7 | 29.0 | 29.5 |
| 8 | 29.6 | 28.2 |
| 9 | 29.5 | 27.5 |
| 10 | 28.5 | 26.5 |
| 11 | 29.3 | 30.9 |
| 12 | 29.4 | 30.3 |
| 13 | 28.7 | 26.2 |
| 14 | 28.7 | 29.7 |
| 15 | 28.9 | 28.1 |
| 16 | 29.3 | 28.3 |
| 17 | 28.8 | 27.4 |
| 18 | 29.7 | 30.0 |
| 19 | 29.3 | 26.6 |
| 20 | 29.1 | 29.1 |
| 21 | 28.7 | 30.6 |
| 22 | 29.2 | 28.6 |
| 23 | 29.1 | 27.3 |
| 24 | 29.1 | 28.2 |
| 25 | 28.7 | 28.7 |
| 26 | 28.8 | 28.9 |
| 27 | 29.0 | 27.7 |
| 28 | 28.8 | 28.4 |
| 29 | 29.6 | 29.8 |
| 30 | 28.9 | 28.5 |
| 31 | 29.4 | 29.0 |
| 32 | 29.0 | 28.5 |
| 33 | 29.6 | 29.9 |
| 34 | 29.5 | 28.1 |
| 35 | 29.2 | 29.4 |
| 36 | 28.9 | 29.3 |
| 37 | 29.5 | 27.9 |
| 38 | 28.6 | 29.4 |
| 39 | 28.6 | 26.4 |
| 40 | 28.8 | 28.8 |
| 41 | 28.8 | 26.7 |
| 42 | 29.1 | 29.1 |
| 43 | 29.3 | 29.1 |
| 44 | 28.9 | 28.7 |
| 45 | 29.4 | 28.8 |
| 46 | 28.3 | 28.2 |
| 47 | 29.0 | 28.6 |
| 48 | 29.1 | 28.0 |
| 49 | 28.8 | 25.6 |
| 50 | 29.9 | 28.9 |
| 51 | 29.1 | 27.5 |
| 52 | 29.6 | 28.5 |
| 53 | 29.4 | 29.4 |
| 54 | 29.2 | 24.7 |
| 55 | 28.9 | 29.9 |
| 56 | 28.8 | 25.1 |
| 57 | 29.4 | 28.6 |
| 58 | 28.6 | 27.9 |
| 59 | 28.8 | 27.1 |
| 60 | 29.7 | 27.3 |
| 61 | 29.2 | 28.0 |
| 62 | 29.4 | 27.4 |
| 63 | 29.6 | 27.3 |
| 64 | 28.6 | 28.0 |
| 65 | 29.2 | 25.9 |
| 66 | 28.8 | 28.1 |
| 67 | 29.3 | 29.4 |
| 68 | 29.8 | 28.7 |
| 69 | 29.3 | 28.9 |
| 70 | 28.7 | 27.3 |
| 71 | 29.2 | 29.1 |
| 72 | 29.7 | 28.9 |
| 73 | 29.1 | 27.4 |
| 74 | 29.1 | 29.0 |
| 75 | 28.6 | 25.8 |
| 76 | 29.4 | 27.6 |
| 77 | 29.0 | 27.5 |
| 78 | 29.3 | 27.4 |
| 79 | 28.8 | 28.7 |
| 80 | 29.2 | 27.0 |
| 81 | 29.6 | 29.4 |
| 82 | 29.3 | 30.2 |
| 83 | 29.3 | 26.6 |
| 84 | 29.2 | 26.9 |
| 85 | 28.7 | 27.4 |
| 86 | 29.5 | 30.5 |
| 87 | 29.6 | 28.5 |
| 88 | 29.1 | 27.9 |
| 89 | 29.2 | 26.4 |
| 90 | 29.0 | 27.6 |
| 91 | 28.8 | 26.3 |
| 92 | 29.3 | 27.9 |
| 93 | 29.2 | 26.3 |
| 94 | 28.5 | 27.9 |
| 95 | 29.5 | 26.6 |
| 96 | 29.6 | 30.2 |
| 97 | 29.2 | 30.1 |
| 98 | 29.8 | 30.1 |
| 99 | 29.0 | 29.9 |
| 100 | 29.3 | 27.8 |
| 101 | 28.8 | 27.6 |
| 102 | 29.3 | 28.6 |

### Example 2

### Comparison of PUP1 and QTL-Based Prediction

The ability of PUP1 to predict phenotypes in predicted populations was compared with conventional QTL-based prediction based on real data of 78 bi-parental F₄ populations from nine (9) reference populations in corn QTL mapping and MAS projects (see Tables 10, 11, and 12 below). The trait of interest was corn moisture, which is one of the most important traits in corn breeding. QTL-based prediction included two steps: (i) QTL markers were identified using marker-based composite interval mapping (Zeng, 1994) with five cofactors selected by forward selection in a reference population based on an empirical LOD threshold estimated from 5000 permutations (Churchill & Doerge, 1994); and (ii) the effects of those QTL markers identified were estimated using multiple regression and used to predict the phenotype of an individual in a predicted population by summing the effects of the QTL markers identified based on the individual's genotype. The prediction method used for PUP1 was that described hereinabove in Section II.A. In the initial comparisons between PUP1 and QTL-based predictions, the influence of genetic similarity on the accuracy of prediction was not considered.

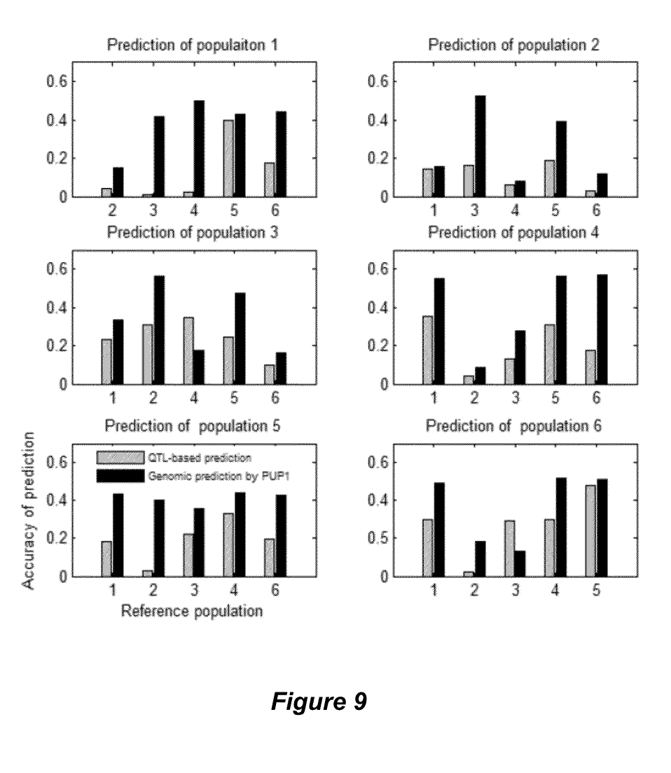

The comparison was established for 78 F₄ populations from nine marker-assisted breeding projects (see Tables 10-12; discussed in more detail hereinbelow regarding use of network populations in PUP2). For the purposes of these comparisons, a network population was established using 7 parents to generate 6 bi-parental subpopulations, all of which were genotyped with respect to a same set of molecular markers. Each subpopulation was treated as a predicted population and predicted in turn by each of the remaining populations. For example, there are six (6) subpopulations in Network 9 (see Table 12 and FIG. 9). To predict phenotypes for subpopulation 1, subpopulations 2, 3, 4, 5, and 6 (see FIG. 9) were used as five different reference populations for this purpose. Similarly, subpopulations 1 and 3-6 were used as reference populations to predict subpopulation 1, subpopulations 1, 2, and 4-6 were used as reference populations to predict subpopulation 3, subpopulations 1-3, 5, and 6 were used as reference populations to predict subpopulation 4, etc.

The project included six bi-parental populations (Network Population 9, subpopulations 1-6; see Table 12). In total, seven different parents were employed to generate six bi-parental populations, and these subpopulations were inter-connected by one common parent (049 in Table 12). The number of polymorphic marker loci used for each population was determined by genotyping the parents using 1200 marker loci and 232 markers that segregated among the parents were used for genotyping. The actual number of polymorphic markers varied from population to population (see Table 12 below). Typically, each of the 232 segregating loci was defined by 1 to 5 SNPs, and the genotype of a locus of a given individual was represented by a combination of the SNPs present at each locus expressed as a haplotype. The genotype of a locus was coded using the method described hereinabove. Each bi-parental population included a plurality of F₄ progeny derived from two inbred parents, which were genotyped and then testcrossed to a tester.

The phenotypic scores with respect to grain moisture were obtained based on hybrids of the F₄ progeny individuals across five locations. The phenotypes were then analyzed using the mixed model of Equation (3) and the BLUP of each progeny individual was employed for the following prediction analysis.

Each individual population was experimentally predicted with respect to phenotype based only on the determined genotypes using the other five individual populations serving as individual reference populations. In these initial experiments, genetic similarity was not used for controlling the selection of a reference population for a given predicted population. QTL-based prediction was used to first identify significant QTL markers using a procedure similar to composite interval mapping (CIM), and then the effects of the markers were calculated by multiple regression in each reference population. In PUP1, the effect of each marker on a genome was calculated using GBLUP (Meuwissen et al., 2001) based on a reference population.

FIG. 9 also shows the more accurate prediction using PUP1 as compared to using QTL-based prediction for six subpopulations in the network. The extent of the increases in the accuracies of the predictions due to PUP1 varied with the predicted and reference populations. This type of trend was shown for other network populations, indicating that PUP1 yielded higher predictive ability than did the QTL-based approach.

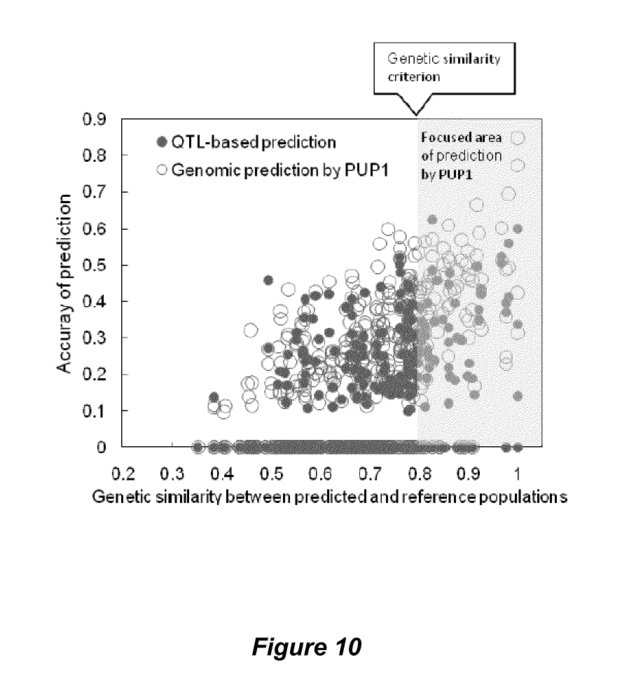

FIG. 10 shows the relationship between the accuracy of prediction and genetic similarity between the predicted and reference populations. The method used for calculating genetic similarities in PUP1 was as set forth in Section II.A.1 above. Specifically, the genetic similarity between a predicted and a reference populations was calculated based on the marker genotypes from the parents used to generate the predicted and reference populations. The accuracies of prediction were expressed as the correlation coefficients between predicted and observed phenotypes. Theoretically, in a network population serving as a reference population composed of n subpopulations, there are [n>(n−1)]×0.5 possible predictions using PUP1, since each population can be predicted (n−1) times by the other individual n−1 subpopulations that make up the network reference population.

Therefore, for the nine networks listed in Tables 10-12, there are 347 predictions for either QTL-based prediction or PUP1. The genetic similarities between reference and predicted population were also calculated along with predictions of each population. In Network 1 of Table 10, subpopulation 1 was employed as a reference population to predict subpopulation 4. To do this, the genetic similarity between subpopulations 1 and 4 was first calculated. Marker genotypes of the four parents used to generate the two subpopulations (i.e., parents 001 and 002 for subpopulation 1 and 003 and 004 for subpopulation 4) are determined. These parents were genotyped using the same set of markers, and it was determined that a total of 263 markers were identified as polymorphic markers for genotyping out of 1200 total markers examined.

Parent 003, which was one of parents employed for generating predicted subpopulation 4, was first examined. Genetic similarities between parent 003 and parent 001 and parent 002 of reference population 1 were determined using the 263 markers as S₀₀₃₋₀₀₁=0.76 and S₀₀₃₋₀₀₂=0.65. Parent 001 was first selected to pair with 003 since it showed a higher genetic similarity than did parent 002. The genetic similarity S₀₀₄₋₀₀₁ between the remaining two parents 004 and 002 was calculated as S₀₀₄₋₀₀₂=0.69. Finally, the average of S₀₀₃₋₀₀₁ and S₀₀₄₋₀₀₂ was calculated as the genetic similarity between subpopulation 1 and 4. Following the similar strategy, the genetic similarities between each pair of subpopulations in each network of Tables 10-12 were determined.

As a result, 347 pairs of predictions and genetic similarities for either QTL-based prediction or PUP1 were plotted in FIG. 10 to clearly the relationships among them across the nine networks studied. For each pair of predictions within each network, there were one predicted population and one reference population. First, the effects of QTL or markers were estimated from the reference population, and then the predicted phenotype of the members of the predicted population were calculated using the estimated effects based on the genotype of the members of the predicted population only. Subsequently, the correlation coefficient between the predicted phenotypes and the real phenotypes from the predicted population was calculated as a measurement of the accuracy of prediction. Overall, for each pair of predictions, one value of genetic similarity and one value of accuracy of prediction were generated.

QTL-based prediction was used to first identify significant QTL markers using a procedure similar to composite interval mapping (CIM: Zeng, 1994), and then the effects of the markers were calculated by multiple regression in a reference population. PUP1 was used to calculate the effect of each marker on a genome using GBLUP (Meuwissen et al., 2001) without the identification of QTL in a reference population. Seventy-eight (78) bi-parental populations from nine (9) network populations were predicted using both methods. The shadowed region of FIG. 10 between 0.8 and 1 on the x-axis represents the focused area of PUP1 wherein the genetic similarity criterion was greater than 0.80. The accuracies increased with the genetic similarities for PUP1 and QTL-based prediction. The higher the genetic similarity was, the better the prediction was. It can be seen that a criterion of genetic similarity could be used to ensure an expected accuracy of prediction. The criterion chosen was 0.8 for PUP1 such that the mean accuracy of the predictions selected by this criterion is equal to 0.40, an increase of 21% compared to 0.33 from the QTL-based predictions (see FIG. 3).

FIG. 9 shows that under some circumstances, QTL-based prediction performed better than PUP1, which can be explained as follows. In PUP1, a single reference population is typically employed. As a consequence, an estimate of the effect of an allele that is only present in a predicted population cannot be provided. By way of example and not limitation, suppose there are two alleles α and β at a QTL locus in a reference population. The effects of α and β can be calculated (e.g., by BLUP) from the population. Next, these effects are employed for predicting phenotypes of a phenotype-unknown population (i.e., a predicted population) with alleles α and γ at the same locus. Under these conditions, the effect of the allele γ cannot be determined because it is not present in the reference population. Consequently, this can lead to a less optimal prediction using PUP1 if the allele γ has a different effect from the allele R.

### Example 3

### Exemplary Implementation of PUP2

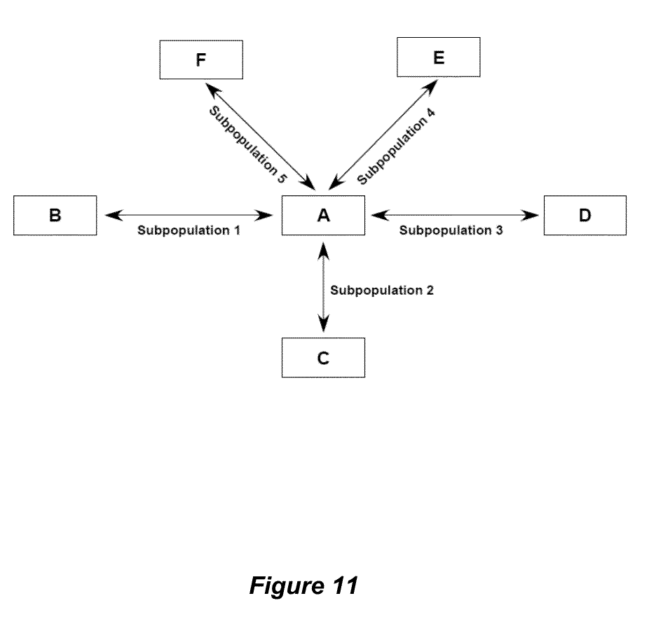

PUP2 was employed to predict the phenotypes of individuals in a predicted population. The reference population employed was a network population composed of five F₄ subpopulations, each of which was derived from two inbred parents (see Table 4). The connection structure among these 5 populations is shown in FIG. 11. Based on parental marker screening, the genetic similarity between the reference and predicted populations was 0.86.

TABLE 4
Summary of Each Subpopulation within the
PUP2 Reference Network Population

| | | | | | Number of |
|---|---|---|---|---|---|
| Subpop. | Female | Male | | | polymorphic |
| No. | parent | parent | Individuals | Markers | markers |
| 1 | A | B | 45 | 232 | 170 |
| 2 | C | A | 97 | 232 | 156 |
| 3 | D | A | 53 | 232 | 132 |
| 4 | E | A | 156 | 232 | 164 |
| 5 | F | A | 103 | 232 | 156 |

The effects of markers were estimated based on genotypic and phenotypic data from the network reference population (see Table 5). These estimates were calculated using Equations (7), (4a), (4b), (4c), and (4d).

TABLE 5
Estimated Marker Effects from the Above Network Reference Population

| | | | Marker Effect |
|---|---|---|---|
| | Locus | Locus Position | Estimated by |
| Chromosome | Name | (cM) | a Network |
| 1 | SM0095 | 6.89 | 0.02 |
| 1 | SM0532 | 44.6 | −0.05 |
| 1 | SM0208 | 47.46 | −0.06 |
| 1 | SM1099 | 49.32 | −0.04 |
| 1 | SM0388 | 53.66 | 0.02 |
| 1 | SM0687 | 60.16 | 0.04 |
| 1 | SM0103 | 65.15 | −0.03 |
| 1 | SM0959 | 91.02 | 0.04 |
| 2 | SM0372 | 31.57 | −0.03 |
| 2 | SM0405 | 35.76 | −0.03 |
| 2 | SM0020 | 50.26 | −0.04 |
| 2 | SM0064 | 52.2 | −0.02 |
| 2 | SM0070 | 54.43 | −0.04 |
| 2 | SM0616 | 63.34 | −0.06 |
| 2 | SM0040 | 66.33 | −0.04 |
| 2 | SM0516 | 67.74 | −0.06 |
| 2 | SM0410 | 89.67 | −0.02 |
| 2 | SM0370 | 90.18 | −0.01 |
| 2 | SM1095 | 91.78 | −0.01 |
| 2 | SM0289 | 96.44 | −0.01 |
| 2 | SM1100 | 98.58 | −0.01 |
| 2 | SM0484 | 132.88 | −0.02 |
| 3 | SM0411 | 33.54 | −0.02 |
| 3 | SM0646 | 50.96 | −0.01 |
| 3 | SM0418 | 69.96 | −0.01 |
| 3 | SM0314 | 93.21 | 0.03 |
| 3 | SM0967 | 101.41 | 0.06 |
| 3 | SM0005 | 106.65 | 0.07 |
| 3 | SM0364 | 113.05 | −0.03 |
| 3 | SM0668 | 114.52 | −0.01 |
| 4 | SM1098 | 49.62 | 0.02 |
| 4 | SM0239 | 65.34 | −0.03 |
| 4 | SM0274 | 72.87 | −0.05 |
| 4 | SM0066 | 92.79 | −0.02 |
| 4 | SM0425 | 100.2 | −0.03 |
| 4 | SM0258 | 102.02 | −0.01 |
| 5 | SM0269 | 27.14 | 0.05 |
| 5 | SM1011 | 37.72 | −0.03 |
| 5 | SM1125 | 43.01 | −0.04 |
| 5 | SM0493 | 73.82 | −0.06 |
| 5 | SM0105 | 74.01 | −0.05 |
| 5 | SM0138 | 77.93 | −0.03 |
| 5 | SM0648 | 80.11 | 0.04 |
| 5 | SM0108 | 82.47 | −0.02 |
| 5 | SM0632 | 86.28 | 0.04 |
| 5 | SM0802 | 88.36 | −0.04 |
| 5 | SM0205 | 91.65 | −0.02 |
| 5 | SM0803 | 96.79 | −0.02 |
| 5 | SM0987 | 104.99 | −0.01 |
| 6 | SM1051 | 17.16 | −0.05 |
| 6 | SM0115 | 21.32 | −0.04 |
| 6 | SM0315 | 30.46 | −0.01 |
| 6 | SM0156 | 37.16 | −0.02 |
| 6 | SM0259 | 84.65 | −0.04 |
| 6 | SM0940 | 85.6 | 0.02 |
| 6 | SM1118 | 91.69 | −0.02 |
| 7 | SM0368 | 0 | 0.05 |
| 7 | SM0904 | 3.05 | 0.03 |
| 7 | SM0358 | 26.77 | −0.02 |
| 7 | SM0359 | 28.1 | −0.03 |
| 7 | SM0122 | 30.45 | −0.02 |
| 7 | SM0093 | 38.48 | −0.03 |
| 7 | SM0014 | 39.47 | −0.02 |
| 7 | SM0077 | 43.72 | −0.02 |
| 7 | SM1015 | 48 | −0.02 |
| 7 | SM0912 | 63.77 | 0.02 |
| 7 | SM0167 | 64.59 | 0.02 |
| 7 | SM0074 | 82.79 | 0.04 |
| 7 | SM0342 | 100 | 0.01 |
| 7 | SM0139 | 101.29 | 0.01 |
| 8 | SM0300 | 0.82 | −0.02 |
| 8 | SM0727 | 7.09 | 0.01 |
| 8 | SM0826 | 19.13 | 0.01 |
| 8 | SM0248 | 28.28 | 0.06 |
| 8 | SM0036 | 42.98 | 0.02 |
| 8 | SM0271 | 65.48 | −0.02 |
| 8 | SM0538 | 99.28 | −0.03 |
| 8 | SM0949 | 102.79 | −0.06 |
| 8 | SM0596 | 105.88 | −0.04 |
| 8 | SM0528 | 107.63 | −0.06 |
| 8 | SM0780 | 109.97 | −0.01 |
| 9 | SM0847 | 23.64 | −0.03 |
| 9 | SM0469 | 25.9 | 0.02 |
| 9 | SM0180 | 30.42 | −0.01 |
| 9 | SM0353 | 38.71 | 0.01 |
| 9 | SM0908 | 96.44 | 0.01 |
| 10 | SM0913 | 16.74 | 0.01 |
| 10 | SM0965 | 24.49 | −0.02 |
| 10 | SM0474 | 25.02 | −0.01 |
| 10 | SM0943 | 49.27 | −0.01 |
| 10 | SM1019 | 55.95 | −0.07 |
| 10 | SM0478 | 58.46 | −0.04 |
| 10 | SM0503 | 67.19 | −0.01 |
| 10 | SM0954 | 76.87 | −0.01 |
| 10 | SM0953 | 77.77 | −0.02 |
| 10 | SM0898 | 78.63 | −0.03 |

Next, the phenotypes of the individuals in the predicted population were predicted based on marker genotypic data using Equation (5). The population included 102 individuals, and each individual was genotyped using 81 SNP markers. The phenotype of each individual in the predicted population was calculated based on the same set of markers for which effects were estimated from the reference population (see Table 6). Table 7 summarizes the predicted grain moistures for the 102 individuals in the predicted population.

TABLE 6
Markers and Calculated Marker Effects Employed for
Phenotype Prediction

| | Marker | Marker | |
|---|---|---|---|
| Chromosome | name | position (cM) | Marker Effects |
| 1 | SM0095C | 6.9 | 0.02 |
| 1 | SM0532B | 44.6 | −0.05 |
| 1 | SM0208B | 47.5 | −0.06 |
| 1 | SM1099B | 49.3 | −0.04 |
| 1 | SM0388B | 53.7 | 0.02 |
| 1 | SM0687C | 60.2 | 0.04 |
| 1 | SM0103A | 65.2 | −0.03 |
| 1 | SM0959B | 91.0 | 0.04 |
| 2 | SM0372B | 31.6 | −0.03 |
| 2 | SM0405C | 35.8 | −0.03 |
| 2 | SM0020C | 50.3 | −0.04 |
| 2 | SM0064A | 52.2 | −0.02 |
| 2 | SM0070C | 54.4 | −0.04 |
| 2 | SM0616A | 63.3 | −0.06 |
| 2 | SM0040B | 66.3 | −0.04 |
| 2 | SM0516A | 67.7 | −0.06 |
| 2 | SM0410D | 89.7 | −0.02 |
| 2 | SM0370A | 90.2 | −0.01 |
| 2 | SM1095A | 91.8 | −0.01 |
| 2 | SM0289B | 96.4 | −0.01 |
| 2 | SM1100A | 98.6 | −0.01 |
| 2 | SM0484A | 132.9 | −0.02 |
| 3 | SM0411D | 33.5 | −0.02 |
| 3 | SM0646D | 51.0 | −0.01 |
| 3 | SM0418A | 70.0 | −0.01 |
| 3 | SM0314B | 93.2 | 0.03 |
| 3 | SM0967A | 101.4 | 0.06 |
| 3 | SM0005B | 106.7 | 0.07 |
| 3 | SM0364B | 113.1 | −0.03 |
| 3 | SM0668H | 114.5 | −0.01 |
| 4 | SM1098E | 49.6 | 0.02 |
| 4 | SM0239A | 65.3 | −0.03 |
| 4 | SM0274A | 72.9 | −0.05 |
| 4 | SM0066B | 92.8 | −0.02 |
| 4 | SM0425A | 100.2 | −0.03 |
| 4 | SM0258B | 102.0 | −0.01 |
| 5 | SM0269B | 27.1 | 0.05 |
| 5 | SM1011F | 37.7 | −0.03 |
| 5 | SM1125A | 43.0 | −0.04 |
| 5 | SM0493B | 73.8 | −0.06 |
| 5 | SM0105C | 74.0 | −0.05 |
| 5 | SM0138B | 77.9 | −0.03 |
| 5 | SM0648A | 80.1 | 0.04 |
| 5 | SM0108C | 82.5 | −0.02 |
| 5 | SM0632H | 86.3 | 0.04 |
| 5 | SM0802B | 88.4 | −0.04 |
| 5 | SM0205B | 91.7 | −0.02 |
| 5 | SM0803D | 96.8 | −0.02 |
| 5 | SM0987C | 105.0 | −0.01 |
| 6 | SM1051D | 17.2 | −0.05 |
| 6 | SM0115E | 21.3 | −0.04 |
| 6 | SM0315B | 30.5 | −0.01 |
| 6 | SM0156B | 37.2 | −0.02 |
| 6 | SM0259C | 84.7 | −0.04 |
| 6 | SM0940E | 85.6 | 0.02 |
| 6 | SM1118C | 91.7 | −0.02 |
| 7 | SM0368A | 0.0 | 0.05 |
| 7 | SM0904D | 3.1 | 0.03 |
| 7 | SM0358B | 26.8 | −0.02 |
| 7 | SM0359F | 28.1 | −0.03 |
| 7 | SM0122C | 30.5 | −0.02 |
| 7 | SM0093B | 38.5 | −0.03 |
| 7 | SM0014F | 39.5 | −0.02 |
| 7 | SM0077A | 43.7 | −0.02 |
| 7 | SM1015D | 48.0 | −0.02 |
| 7 | SM0912D | 63.8 | 0.02 |
| 7 | SM0167B | 64.6 | 0.02 |
| 7 | SM0074D | 82.8 | 0.04 |
| 7 | SM0342C | 100.0 | 0.01 |
| 7 | SM0139B | 101.3 | 0.01 |
| 8 | SM0300B | 0.8 | −0.02 |
| 8 | SM0727B | 7.1 | 0.01 |
| 8 | SM0826B | 19.1 | 0.01 |
| 8 | SM0248D | 28.3 | 0.06 |
| 8 | SM0036B | 43.0 | 0.02 |
| 8 | SM0271A | 65.5 | −0.02 |
| 8 | SM0538A | 99.3 | −0.03 |
| 8 | SM0949C | 102.8 | −0.06 |
| 8 | SM0596E | 105.9 | −0.04 |
| 8 | SM0528B | 107.6 | −0.06 |
| 8 | SM0780C | 110.0 | −0.01 |
| 9 | SM0847C | 23.6 | −0.03 |
| 9 | SM0469A | 25.9 | 0.02 |
| 9 | SM0180A | 30.4 | −0.01 |
| 9 | SM0353A | 38.7 | 0.01 |
| 9 | SM0908B | 96.4 | 0.01 |
| 10 | SM0913B | 16.7 | 0.01 |
| 10 | SM0965H | 24.5 | −0.02 |
| 10 | SM0965G | 24.5 | −0.02 |
| 10 | SM0474B | 25.0 | −0.01 |
| 10 | SM0943B | 49.3 | −0.01 |
| 10 | SM1019B | 56.0 | −0.07 |
| 10 | SM0478A | 58.5 | −0.04 |
| 10 | SM0503B | 67.2 | −0.01 |
| 10 | SM0954B | 76.9 | −0.01 |
| 10 | SM0953C | 77.8 | −0.02 |
| 10 | SM0898A | 78.6 | −0.03 |

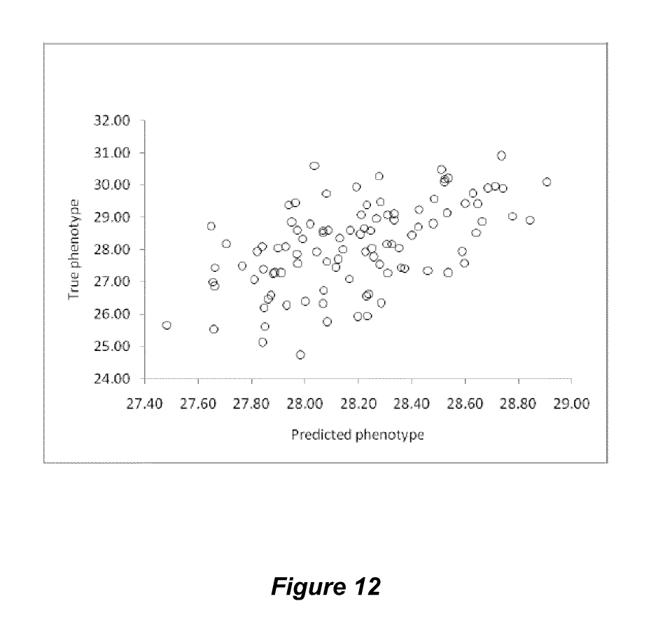

To evaluate the accuracy of prediction using PUP2, grain moisture data were collected across the same locations used in the reference population (see Table 7). The accuracy of prediction was expressed as the correlation coefficient between the predicted phenotypes in a predicted population and actually observed phenotypes in that same predicted population. The accuracy of prediction was 0.56 (see FIG. 12).

TABLE 7
Predicted and Observed Grain Moisture in a Predicted Corn Population

| Individual | Predicted | Observed |
|---|---|---|
| No. | Grain Moisture | Grain Moisture |
| 1 | 27.66 | 27.44 |
| 2 | 27.66 | 25.53 |
| 3 | 28.23 | 25.94 |
| 4 | 27.48 | 25.67 |
| 5 | 27.88 | 27.26 |
| 6 | 28.48 | 29.57 |
| 7 | 28.28 | 29.48 |
| 8 | 28.31 | 28.17 |
| 9 | 28.28 | 27.54 |
| 10 | 27.86 | 26.47 |
| 11 | 28.74 | 30.92 |
| 12 | 28.28 | 30.27 |
| 13 | 27.85 | 26.20 |
| 14 | 28.08 | 29.74 |
| 15 | 27.84 | 28.10 |
| 16 | 27.99 | 28.33 |
| 17 | 27.84 | 27.39 |
| 18 | 28.71 | 29.98 |
| 19 | 28.23 | 26.57 |
| 20 | 28.31 | 29.08 |
| 21 | 28.04 | 30.60 |
| 22 | 27.97 | 28.60 |
| 23 | 27.89 | 27.29 |
| 24 | 28.33 | 28.17 |
| 25 | 27.65 | 28.74 |
| 26 | 27.95 | 28.86 |
| 27 | 28.12 | 27.71 |
| 28 | 28.13 | 28.36 |
| 29 | 28.63 | 29.75 |
| 30 | 28.40 | 28.45 |
| 31 | 28.78 | 29.04 |
| 32 | 28.07 | 28.52 |
| 33 | 28.68 | 29.91 |
| 34 | 28.35 | 28.05 |
| 35 | 27.94 | 29.39 |
| 36 | 28.43 | 29.25 |
| 37 | 28.59 | 27.95 |
| 38 | 27.96 | 29.45 |
| 39 | 28.00 | 26.40 |
| 40 | 28.02 | 28.81 |
| 41 | 28.07 | 26.74 |
| 42 | 28.33 | 29.12 |
| 43 | 28.53 | 29.14 |
| 44 | 28.22 | 28.65 |
| 45 | 28.48 | 28.81 |
| 46 | 27.70 | 28.18 |
| 47 | 28.09 | 28.60 |
| 48 | 28.25 | 28.04 |
| 49 | 27.85 | 25.61 |
| 50 | 28.84 | 28.92 |
| 51 | 28.12 | 27.46 |
| 52 | 28.21 | 28.49 |
| 53 | 28.23 | 29.39 |
| 54 | 27.98 | 24.74 |
| 55 | 28.74 | 29.90 |
| 56 | 27.84 | 25.13 |
| 57 | 28.25 | 28.58 |
| 58 | 27.97 | 27.86 |
| 59 | 28.17 | 27.08 |
| 60 | 28.31 | 27.28 |
| 61 | 28.14 | 28.01 |
| 62 | 28.37 | 27.42 |
| 63 | 28.54 | 27.29 |
| 64 | 27.90 | 28.05 |
| 65 | 28.20 | 25.93 |
| 66 | 27.93 | 28.10 |
| 67 | 28.60 | 29.44 |
| 68 | 28.42 | 28.71 |
| 69 | 28.66 | 28.87 |
| 70 | 27.91 | 27.29 |
| 71 | 28.21 | 29.08 |
| 72 | 28.33 | 28.92 |
| 73 | 27.81 | 27.08 |
| 74 | 28.27 | 28.97 |
| 75 | 28.08 | 25.77 |
| 76 | 28.60 | 27.58 |
| 77 | 27.76 | 27.50 |
| 78 | 28.36 | 27.44 |
| 79 | 28.17 | 28.60 |
| 80 | 27.65 | 26.99 |
| 81 | 28.65 | 29.42 |
| 82 | 28.54 | 30.21 |
| 83 | 27.87 | 26.59 |
| 84 | 27.66 | 26.86 |
| 85 | 28.46 | 27.35 |
| 86 | 28.51 | 30.49 |
| 87 | 28.64 | 28.52 |
| 88 | 28.23 | 27.94 |
| 89 | 28.29 | 26.36 |
| 90 | 27.97 | 27.57 |
| 91 | 28.07 | 26.33 |
| 92 | 28.04 | 27.93 |
| 93 | 27.93 | 26.28 |
| 94 | 27.82 | 27.94 |
| 95 | 28.24 | 26.63 |
| 96 | 28.52 | 30.18 |
| 97 | 28.52 | 30.10 |
| 98 | 28.91 | 30.10 |
| 99 | 28.19 | 29.95 |
| 100 | 28.26 | 27.78 |
| 101 | 28.08 | 27.63 |
| 102 | 28.07 | 28.59 |

### Example 4

### Accuracy of Prediction by PUP2

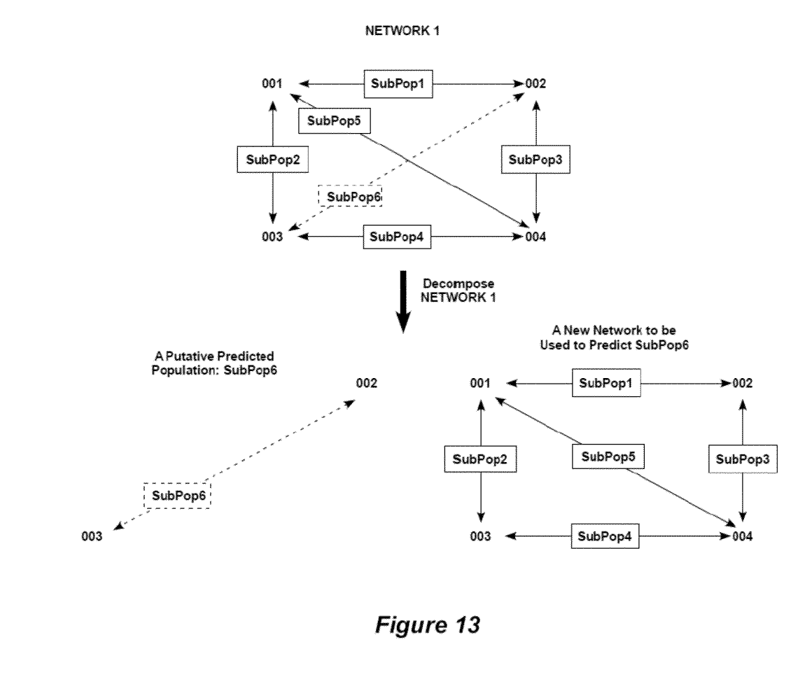

To test the accuracy of PUP2, a complete network was decomposed into a predicted or tested population (see SubPop6 of Table 10), and a new network that included the remaining populations (i.e., SubPop1-SubPop5). The phenotype of a progeny in SubPop6 was predicted by the new network and the accuracy of prediction was calculated as the correlation coefficient between predicted and observed phenotypes in SubPop6. In either Network 1 or the new network, Parents 001, 002, 003, and 004 were four different inbred parents used to generate SubPop1, SubPop2, SubPop3, SubPop4, SubPop5, and SubPop6 (see FIG. 13 and Table 10). Each population was an F₄ population derived from two of the listed inbred parents as indicated in FIG. 13. For each population, a cross between two parents was employed to generate an F₁. The F₁ was selfed to generate an F₂, which itself was selfed to generate an F₃. Finally, the F₄ was obtained by selfing the F₃. By following this basic strategy, each subpopulation within each of nine networks was predicted by a new network that included the rest of the subpopulations within the same network serving as reference populations. Detailed information about these network and population such as female and male used for generating the populations, the number of progeny, and the number of markers used for network and individual populations can be easily found in Tables 10-12. For each population, the phenotypes of each individual with respect to corn moisture were predicted using a different set of markers, depending on networks (see Tables 10-12). Since all the progenies in individual populations within a network were phenotyped across a same set of locations, for simplicity, the phenotypes employed were the BLUPs of the progenies across multiple locations.

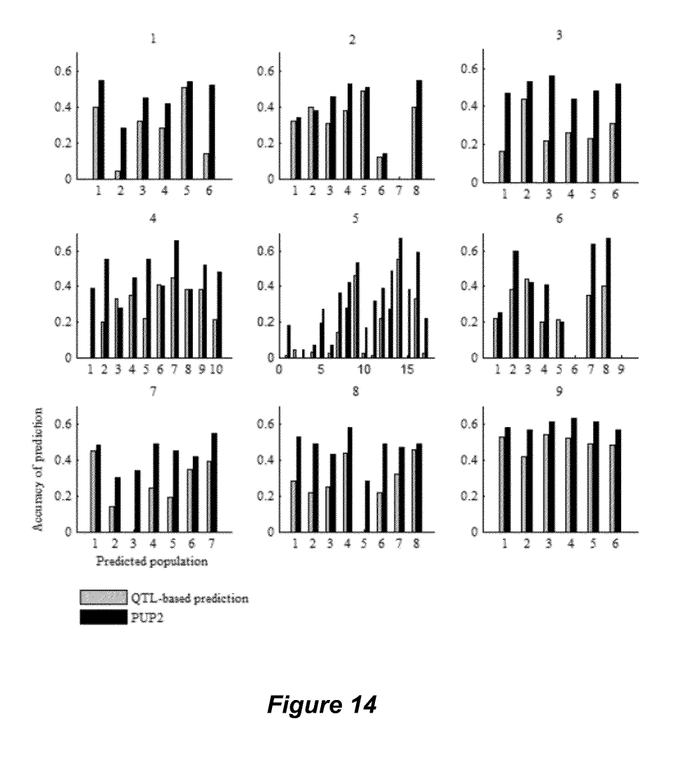

To compare PUP2 to QTL-based predictions, QTLs were used to predict subpopulations as described hereinabove in EXAMPLE 1. As shown in FIG. 14, PUP2 showed more accurate prediction than QTL-based prediction. It was determined that the accuracies of the predictions due to PUP2 for 78 subpopulations from 9 networks were higher than those resulting from QTL-based predictions, except that QTL-based predictions were slightly better than PUP2 in two specific subpopulations (see FIG. 14). These two specific subpopulations were further studied and it was determined that there were one or two large-effect QTLs associated with corn moisture. This suggested that the QTLs captured by GBLUP other than these large-effect QTLs had strong QTLs by genetic background interactions and this type of population-specific interactions reduced the ability of prediction using GBLUP.

Generally, PUP2 also provided superior accuracy of prediction to PUP1. It was determined that the accuracies of the predictions with PUP2 for 6 subpopulations from Network 9 were higher than those resulting from PUP1 (see

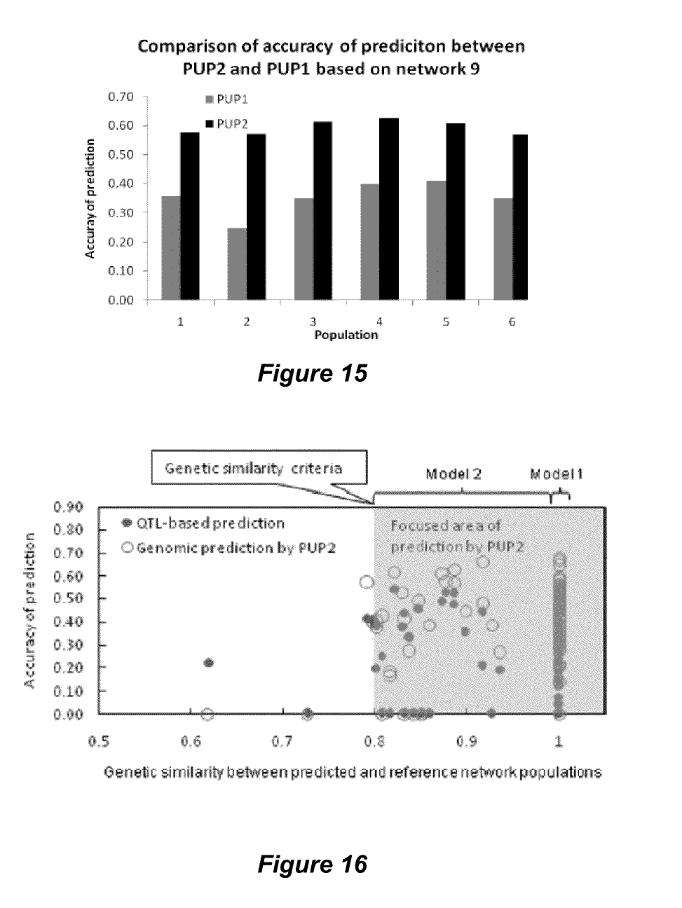

FIG. 15). With PUP1, the phenotype of each individual population was experimentally predicted using the other five populations individually serving as reference populations (i.e., five predictions based on genotype alone for each of the six populations). The accuracy of prediction for a population was calculated as the average of the accuracies across the five predictions produced by the other individual populations. In contrast, with PUP2, a population was predicted by a network composed of the other five individual populations (i.e., the reference population considered the give subpopulations cumulatively rather than individually). In both PUP1 and PUP2, the accuracy of prediction was measured as the correlation coefficient between predicted and observed phenotypes in a predicted population. On average, the accuracies of the predictions with PUP2 increased 65% over those with PUP1. A similar trend was observed for other networks.

Additionally, PUP2 provided more stable predictions than did PUP1. For example, for Network 9, when Population 1 was predicted by each of Populations 2, 3, 4, 5 and 6 individually under the PUP1 approach, the prediction varied with the reference populations from 0.15 to 0.52. This indicated that the accuracies really depended on the selection of a reference population, and were unstable. A high accuracy could be achieved if an appropriate reference population was used. Otherwise, the accuracy could be very low. In contrast, a more stable prediction of 0.59 was obtained from PUP2.

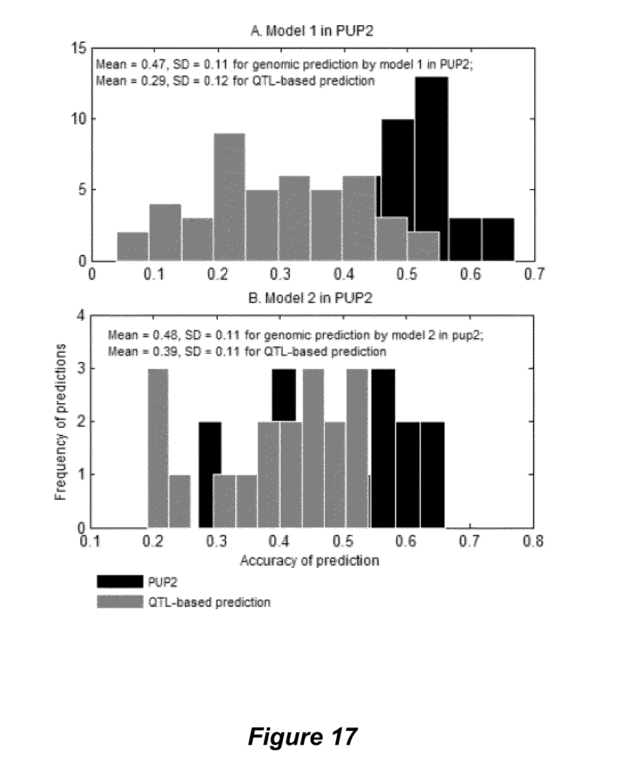

High genetic similarity yielded more accuracy of prediction in PUP2. This was seen for both Model 1 and Model 2 (see FIG. 16). For Model 1, the genetic similarity between predicted and reference network populations was always 1.00 since two parents of the predicted population were already included in the reference population. An empirical similarity of 0.8 was then selected to be the criterion for choosing a reference network population in subsequent analyses. Given this criterion, the mean accuracy of prediction provided by Model 1 in PUP2 was 0.47, which represented an increase of 67% over QTL-based predictions (0.29; see FIG. 17). The same trend was also observed with respect to Model 2.

The significant gain in accuracy of prediction of PUP2 over traditional QTL-based prediction was observed based on real data analysis. There are at least two reasons for this. First, PUP2 is designed to include more QTL in the prediction system than QTL-based prediction systems, the latter of which utilize only significant QTL markers. Second, it is also possible to utilize the genetic variation from QTL by QTL interactions when a whole genome is used for selection as a combination of all the QTL.

The gain of PUP2 over PUP1 can depend on the extent of allelic diversity in the reference population. For example, it would be expected to be difficult to accurately predict a phenotype in a progeny for which a QTL allele was not included in a reference population. Conversely, accuracy of prediction can increase with the diversity of alleles in a network. As such, it is reasonable to employ multiple diverse parents to generate network populations assume in order to maximize the allelic diversity therein.

### Example 5

### Exemplary Implementation of PUP3

PUP3 was employed to predict the phenotypes of a predicted population. The reference population employed to estimate marker effects was a linkage disequilibrium (LD) panel (i.e., a collection of individual germplasm that includes a plurality of inbred germplasms). The LD panel included 585 corn inbred lines, and each line in the LD panel was genotyped with respect to about 20,000 SNP markers.

A best subset of markers was identified using the method of selection described hereinabove in Section II.C. It was determined that an informative subset of 3000 SNP markers could be employed for prediction. Next, the effect of each marker was estimated based on genotypic and phenotypic data of grain yield in the LD panel using the Equations (4), (4a), (4b), (4c), and 4d, and the estimates for 100 of the 3000 SNP markers are shown in Table 8.

TABLE 8
Marker Effects Estimated from a Corn LD Panel

| Marker | Marker | Marker |
|---|---|---|
| No. | Name | Effect |
| 1 | SX3609352 | 0.00 |
| 2 | SX4523970 | 0.01 |
| 3 | SX15539566 | 0.00 |
| 4 | SX15539603 | 0.02 |
| 5 | SX15542934 | 0.00 |
| 6 | SX15542983 | 0.02 |
| 7 | SX15545449 | 0.01 |
| 8 | SX15545491 | 0.00 |
| 9 | SX4789404 | 0.03 |
| 10 | SX4784548 | 0.00 |
| 11 | SX13437169 | 0.03 |
| 12 | SX13437171 | 0.00 |
| 13 | SX13437202 | 0.00 |
| 14 | SX13437213 | 0.00 |
| 15 | SX13438476 | 0.00 |
| 16 | SX4026025 | 0.00 |
| 17 | SX4029449 | 0.01 |
| 18 | SX4028275 | −0.02 |
| 19 | SX4028330 | −0.04 |
| 20 | SX4028397 | 0.01 |
| 21 | SX4950655 | 0.01 |
| 22 | SX4951069 | 0.00 |
| 23 | SX4951398 | 0.02 |
| 24 | SX4951411 | 0.01 |
| 25 | SX6498867 | 0.00 |
| 26 | SX6499053 | 0.03 |
| 27 | SX6499093 | 0.00 |
| 28 | SX4485579 | 0.03 |
| 29 | SX4486424 | 0.02 |
| 30 | SX4486874 | 0.02 |
| 31 | SX4489113 | 0.02 |
| 32 | SX4489119 | 0.02 |
| 33 | SX4489302 | 0.03 |
| 34 | SX3243873 | 0.03 |
| 35 | SX3247177 | 0.03 |
| 36 | SX3247218 | 0.03 |
| 37 | SX4855973 | 0.03 |
| 38 | SX4856144 | 0.00 |
| 39 | SX2807979 | 0.00 |
| 40 | SX2807601 | 0.00 |
| 41 | SX2807341 | 0.00 |
| 42 | SX2807317 | 0.00 |
| 43 | SX2807206 | 0.02 |
| 44 | SX2807196 | 0.00 |
| 45 | SX2806796 | 0.00 |
| 46 | SX2806667 | 0.00 |
| 47 | SX17191575 | 0.00 |
| 48 | SX17191581 | −0.02 |
| 49 | SX17191599 | 0.02 |
| 50 | SX2971993 | −0.03 |
| 51 | SX2972292 | 0.00 |
| 52 | SX2759276 | 0.00 |
| 53 | SX2893920 | 0.01 |
| 54 | SX2894279 | 0.00 |
| 55 | SX2894600 | 0.00 |
| 56 | SX2830700 | 0.00 |
| 57 | SX2830509 | 0.01 |
| 58 | SX2829199 | 0.00 |
| 59 | SX2827713 | 0.01 |
| 60 | SX2826410 | 0.00 |
| 61 | SX16009902 | 0.02 |
| 62 | SX16009959 | 0.01 |
| 63 | SX16010279 | 0.00 |
| 64 | SX16011279 | 0.03 |
| 65 | SX5656865 | 0.00 |
| 66 | SX5657337 | 0.04 |
| 67 | SX5658150 | 0.00 |
| 68 | SX5656232 | −0.02 |
| 69 | SX3374292 | 0.00 |
| 70 | SX3374911 | 0.00 |
| 71 | SX3369008 | 0.00 |
| 72 | SX3369056 | 0.01 |
| 73 | SX3369058 | −0.01 |
| 74 | SX5326026 | 0.00 |
| 75 | SX5325969 | 0.00 |
| 76 | SX5325060 | 0.00 |
| 77 | SX5752872 | 0.01 |
| 78 | SX5752858 | 0.02 |
| 79 | SX5752840 | 0.00 |
| 80 | SX4686974 | 0.04 |
| 81 | SX4686943 | 0.01 |
| 82 | SX4686928 | 0.00 |
| 83 | SX4686923 | 0.01 |
| 84 | SX4685951 | 0.01 |
| 85 | SX4685922 | 0.04 |
| 86 | SX4684871 | 0.02 |
| 87 | SX4684718 | −0.01 |
| 88 | SX2858814 | 0.02 |
| 89 | SX2998083 | 0.01 |
| 90 | SX15637877 | 0.01 |
| 91 | SX5124222 | −0.02 |
| 92 | SX5124679 | 0.03 |
| 93 | SX5125041 | 0.00 |
| 94 | SX2782820 | 0.00 |
| 95 | SX2783780 | 0.00 |
| 96 | SX9194219 | 0.02 |
| 97 | SX9197494 | 0.00 |
| 98 | SX6055655 | 0.00 |
| 99 | SX6055024 | 0.03 |
| 100 | SX6054617 | −0.01 |

A simulated F₄ predicted population derived from a simulated cross of lines 35 and 100 of the LD panel was generated, and 150 simulated genomes of the F₄ predicted population were genotyped with respect to 3000 selected SNP markers. The phenotype predicted for each of the 150 simulated genomes of the predicted population was determined based on genotypic information using Equation (5). See Table 9.

TABLE 9
Predicted Grain Moisture for a PUP-predicted Population

| | Predicted |
|---|---|
| Individual | Grain |
| No. | Moisture |
| 1 | 29.54 |
| 2 | 28.52 |
| 3 | 31.20 |
| 4 | 30.78 |
| 5 | 31.20 |
| 6 | 28.38 |
| 7 | 29.06 |
| 8 | 29.30 |
| 9 | 30.50 |
| 10 | 26.96 |
| 11 | 28.10 |
| 12 | 29.30 |
| 13 | 28.26 |
| 14 | 28.68 |
| 15 | 26.58 |
| 16 | 27.52 |
| 17 | 29.06 |
| 18 | 28.50 |
| 19 | 27.14 |
| 20 | 28.20 |
| 21 | 29.24 |
| 22 | 28.16 |
| 23 | 30.06 |
| 24 | 30.88 |
| 25 | 29.50 |
| 26 | 28.28 |
| 27 | 30.86 |
| 28 | 30.84 |
| 29 | 29.26 |
| 30 | 27.80 |
| 31 | 29.40 |
| 32 | 31.62 |
| 33 | 29.42 |
| 34 | 27.40 |
| 35 | 27.20 |
| 36 | 28.26 |
| 37 | 29.10 |
| 38 | 27.28 |
| 39 | 29.00 |
| 40 | 30.96 |
| 41 | 31.16 |
| 42 | 28.64 |
| 43 | 29.60 |
| 44 | 27.86 |
| 45 | 31.30 |
| 46 | 31.18 |
| 47 | 31.04 |
| 48 | 27.28 |
| 49 | 30.34 |
| 50 | 32.00 |
| 51 | 30.74 |
| 52 | 29.68 |
| 53 | 29.26 |
| 54 | 28.60 |
| 55 | 27.00 |
| 56 | 31.96 |
| 57 | 28.06 |
| 58 | 31.48 |
| 59 | 29.68 |
| 60 | 31.38 |
| 61 | 31.72 |
| 62 | 29.34 |
| 63 | 32.00 |
| 64 | 30.14 |
| 65 | 28.20 |
| 66 | 30.16 |
| 67 | 32.38 |
| 68 | 31.94 |
| 69 | 30.06 |
| 70 | 29.18 |
| 71 | 30.64 |
| 72 | 29.30 |
| 73 | 30.52 |
| 74 | 28.28 |
| 75 | 30.90 |
| 76 | 31.42 |
| 77 | 30.24 |
| 78 | 28.14 |
| 79 | 30.64 |
| 80 | 30.82 |
| 81 | 31.22 |
| 82 | 30.94 |
| 83 | 28.62 |
| 84 | 31.92 |
| 85 | 30.42 |
| 86 | 29.10 |
| 87 | 28.98 |
| 88 | 28.74 |
| 89 | 28.90 |
| 90 | 31.74 |
| 91 | 30.90 |
| 92 | 27.66 |
| 93 | 30.04 |
| 94 | 28.74 |
| 95 | 29.18 |
| 96 | 28.94 |
| 97 | 30.16 |
| 98 | 30.52 |
| 99 | 32.78 |
| 100 | 27.68 |
| 101 | 27.72 |
| 102 | 29.80 |
| 103 | 28.44 |
| 104 | 29.22 |
| 105 | 29.12 |
| 106 | 29.62 |
| 107 | 30.60 |
| 108 | 31.16 |
| 109 | 28.28 |
| 110 | 29.80 |
| 111 | 31.50 |
| 112 | 28.20 |
| 113 | 28.98 |
| 114 | 28.78 |
| 115 | 27.54 |
| 116 | 31.16 |
| 117 | 28.58 |
| 118 | 31.58 |
| 119 | 27.90 |
| 120 | 30.18 |
| 121 | 31.00 |
| 122 | 28.74 |
| 123 | 31.88 |
| 124 | 28.02 |
| 125 | 30.90 |
| 126 | 31.40 |
| 127 | 30.86 |
| 128 | 28.26 |
| 129 | 30.54 |
| 130 | 31.68 |
| 131 | 26.08 |
| 132 | 28.02 |
| 133 | 30.40 |
| 134 | 30.08 |
| 135 | 27.98 |
| 136 | 32.20 |
| 137 | 30.14 |
| 138 | 28.32 |
| 139 | 28.48 |
| 140 | 31.28 |
| 141 | 32.72 |
| 142 | 30.98 |
| 143 | 30.34 |
| 144 | 30.28 |
| 145 | 30.16 |
| 146 | 28.26 |
| 147 | 29.02 |
| 148 | 32.70 |
| 149 | 31.92 |
| 150 | 29.68 |

### Discussion of the Examples

It is believed that the approaches disclosed herein differ from previously disclosed research in plant breeding (see e.g., Jannink et al., 2010). For example, genomic selection to date has only been applied to predict progeny within a breeding population (see e.g., Rex & Yu, 2007; Jannink et al., 2010). In contrast, the methods disclosed herein can employ information determined from previous breeding populations and/or from different locations and/or growing seasons to predict a phenotype in a progeny individual based only on genotypic data. As such, the presently disclosed subject matter provides what is believed to be the first application of genomic prediction in the field of plant breeding.

Advantages of the compositions and methods disclosed herein include at least the following. First, they provide time- and cost-efficient breeding strategies developed specifically for plant breeding. Superior progeny can be selected based only on genotypic marker data with no need for the time, expense, effort, and resources required for phenotyping numerous progeny individuals, which means that selection of desirable lines and/or breeding partners can be performed very early in a breeding project.

Second, the methods disclosed herein allow for the combining of three types of breeding resources to increase genetic gain: (i) typical bi-parental populations; (ii) advanced network populations that can include several or many bi-parental populations; and (iii) LD panels comprising several to many current elite lines.

Third, a higher accuracy of prediction is expected from employing the compositions and methods disclosed herein due at least in part to introducing consideration of genetic similarity among members of the reference population(s) and/or the parents employed to generate the predicted populations, which facilitates selectively choosing one or more desirable reference populations upon which the analyses can be based. Thus, considering the genetic similarity between reference and predicted populations can enhance the ultimate prediction, especially when the interactions between QTL and different genetic backgrounds are considered.

And finally, rather than using all high density markers for prediction, the presently disclosed subject matter relates in some embodiments to methods for combining simple marker regression, genomic best linear unbiased prediction, and cross validation to identify one or more subsets of optimal markers that can yield superior predictions. The use of an optimal marker set can result in cost and time savings without drastically reducing the accuracy of the prediction.

### REFERENCES

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
Allard (1960) Principles of Plant Breeding, John Wiley & Sons, New York, N.Y., United States of America, pages 50-98.
Altschul et al. (1990) Basic local alignment search tool. J Mol Biol 215:403-410.
Altschul et al. (1997) Gapped BLAST and PSI-BLAST: A new generation of protein database search programs. Nucl Acids Res 25:3389-3402.
Ausubel et al. (eds.) (1999) Short Protocols in Molecular Biology Wiley, New York, N.Y., United States of America.
Beavis (1997) “QTL analyses: power, precision, and accuracy, have missing genotypes at the marker”, in Molecular Dissection of Complex Traits Paterson (ed.) CRC Press, New York, N.Y., United States of America.
Bernardo & Yu (2007) Prospects for genome-wide selection for quantitative traits in maize. Crop Science 47:1082-1090.
Delvin & Risch (1995) A comparison of linkage disequilibrium measures for fine-scale mapping. Genomics 29:311-322.
Hayes et al. (2009) Invited review: Genomic selection in dairy cattle: Progress and challenges. Journal of Dairy Science 92:433-443.
Henderson, C R (1975) Best Linear Unbiased Estimation and Prediction under a Selection Model. Biometrics 31 (2): 423-448.
Hocking, R. R. (1976) The Analysis and Selection of Variables in Linear Regression. Biometrics, 32
Hospital et al. (1997) More on the efficiency of marker-assisted selection. Theoretical and Applied Genetics 95:1181-1189.
Jannink et al. (2010) Genomic selection in plant breeding: from theory to practice. Briefings in Functional Genomics 9:166-177.
Jorde (2000) Linkage disequilibrium and the search for complex disease genes. Genome Res 10:1435-1444.
Lande & Thompson (1990) Efficiency of marker-assisted selection in the improvement of quantitative traits. Genetics 124:743-756.
Larkin et al. (2007). Clustal W and Clustal X version 2.0. Bioinformatics, 23:2947-2948.
Legarra et al. (2008) Performance of genomic selection in mice. Genetics 180: 611-618.
Lui, Ben Hui (1998) Statistical Genomics: Linkage, Mapping and QTL Analysis. Page 402-405.
Meuwissen, Hayes and Goddard (2001) Prediction of total genetic value using genome-wide dense marker maps. Genetics 157: 1819-1829
Meuwissen & Goddard (2010) Accurate prediction of genetic values for complex traits by whole genome resequencing. Genetics (2010 Mar. 22. [Epub ahead of print]).
Nei (1978) Estimation of Average Heterozygosity and Genetic Distance from a Small Number of Individuals. Genetics 89:583-590.
Nei & Roychoudhury (1974) Sampling variances of heterozygosity and genetic distance. Genetics 76:379-390.
Tijssen (1993) in Laboratory Techniques in Biochemistry and Molecular Biology, Elsevier, New York, N.Y., United States of America.
Yang et al. (2010) Genetic analysis and characterization of a new maize association mapping panel for quantitative trait loci dissection. Theoretical and Applied Genetics (2010 Mar. 27. [Epub ahead of print]).
Zeng (1994) Precision Mapping of Quantitative Trait Loci. Genetics 136:1457-1468.

It will be understood that various details of the presently disclosed subject matter can be changed without departing from the scope of the presently disclosed subject matter. Furthermore, the foregoing description is for the purpose of illustration only, and not for the purpose of limitation.

TABLE 10
Basic Information for Network Populations 1 to 4*

| Network | Number of | Sub | | | | Number | Number | Number of |
|---|---|---|---|---|---|---|---|---|
| Population | Sub | Population | Female | Male | Generation | of | of | Polymorphic |
| Number | Populations | Number | Parent | Parent | Analyzed | Progeny | Markers | Markers |
| 1 | 6 | 1 | 001 | 002 | F₄ | 178 | 263 | 173 |
| | | 2 | 003 | 001 | F₄ | 177 | 263 | 164 |
| | | 3 | 002 | 004 | F₄ | 176 | 263 | 177 |
| | | 4 | 003 | 004 | F₄ | 176 | 263 | 171 |
| | | 5 | 003 | 002 | F₄ | 171 | 283 | 161 |
| | | 6 | 004 | 001 | F₄ | 180 | 263 | 163 |
| 2 | 9 | 1 | 005 | 006 | F₄ | 180 | 284 | 126 |
| | | 2 | 007 | 006 | F₄ | 171 | 284 | 126 |
| | | 3 | 007 | 005 | F₄ | 180 | 284 | 121 |
| | | 4 | 002 | 008 | F₄ | 178 | 284 | 179 |
| | | 5 | 002 | 006 | F₄ | 171 | 284 | 226 |
| | | 6 | 002 | 005 | F₄ | 149 | 284 | 213 |
| | | 7 | 005 | 005 | F₄ | 174 | 284 | 133 |
| | | 8 | 007 | 002 | F₄ | 175 | 284 | 185 |
| | | 9 | 007 | 008 | F₄ | 176 | 284 | 96 |
| 3 | 6 | 1 | 009 | 010 | F₄ | 180 | 241 | 125 |
| | | 2 | 009 | 011 | F₄ | 180 | 241 | 131 |
| | | 3 | 010 | 011 | F₄ | 170 | 241 | 172 |
| | | 4 | 012 | 009 | F₄ | 113 | 241 | 88 |
| | | 5 | 012 | 010 | F₄ | 180 | 241 | 155 |
| | | 6 | 012 | 011 | F₄ | 180 | 241 | 138 |
| 4 | 10 | 1 | 002 | 013 | F₄ | 85 | 217 | 148 |
| | | 2 | 002 | 014 | F₄ | 102 | 217 | 164 |
| | | 3 | 015 | 008 | F₄ | 89 | 217 | 106 |
| | | 4 | 014 | 016 | F₄ | 77 | 217 | 140 |
| | | 5 | 014 | 008 | F₄ | 91 | 217 | 144 |
| | | 6 | 014 | 017 | F₄ | 87 | 217 | 136 |
| | | 7 | 018 | 014 | F₄ | 115 | 217 | 164 |
| | | 8 | 001 | 007 | F₄ | 86 | 217 | 118 |
| | | 9 | 001 | 019 | F₄ | 102 | 217 | 127 |
| | | 10 | 020 | 021 | F₄ | 91 | 217 | 163 |

*Each network population disclosed in Tables 1-3 is composed of several sub bi-parental population with common parents.
Note
that the number of markers represents the number of all the polymorphic markers in a network population, while the number of polymorphic markers shows the number of markers which segregate in only one sub population.

TABLE 11
Basic Information for Network populations 5 to 7

| Network | Number of | Sub | | | | Number | Number | Number of |
|---|---|---|---|---|---|---|---|---|
| Population | Sub | Population | Female | Male | Generation | of | of | Polymorphic |
| Number | Populations | Number | Parent | Parent | Analyzed | Progeny | Markers | Markers |
| 5 | 17 | 1 | 022 | 023 | F₄ | 131 | 218 | 116 |
| | | 2 | 002 | 004 | F₄ | 94 | 215 | 138 |
| | | 3 | 024 | 025 | F₄ | 101 | 218 | 83 |
| | | 4 | 026 | 027 | F₄ | 57 | 218 | 117 |
| | | 5 | 028 | 029 | F₄ | 88 | 218 | 114 |
| | | 6 | 030 | 031 | F₄ | 49 | 218 | 99 |
| | | 7 | 030 | 022 | F₄ | 123 | 218 | 137 |
| | | 8 | 030 | 029 | F₄ | 84 | 218 | 115 |
| | | 9 | 025 | 022 | F₄ | 39 | 218 | 151 |
| | | 10 | 025 | 001 | F₄ | 89 | 218 | 116 |
| | | 11 | 004 | 022 | F₄ | 46 | 218 | 137 |
| | | 12 | 032 | 002 | F₄ | 44 | 218 | 136 |
| | | 13 | 032 | 033 | F₄ | 49 | 218 | 136 |
| | | 14 | 034 | 022 | F₄ | 51 | 218 | 137 |
| | | 15 | 034 | 035 | F₄ | 47 | 218 | 140 |
| | | 16 | 036 | 022 | F₄ | 78 | 218 | 137 |
| | | 17 | 036 | 037 | F₄ | 47 | 218 | 139 |
| 6 | 9 | 1 | 022 | 038 | F₄ | 54 | 200 | 125 |
| | | 2 | 002 | 039 | F₄ | 61 | 209 | 97 |
| | | 3 | 002 | 023 | F₄ | 85 | 209 | 114 |
| | | 4 | 002 | 040 | F₄ | 64 | 209 | 125 |
| | | 5 | 002 | 039 | F₄ | 118 | 209 | 97 |
| | | 6 | 041 | 039 | F₄ | 83 | 209 | 60 |
| | | 7 | 033 | 002 | F₄ | 123 | 209 | 124 |
| | | 8 | 033 | 002 | F₄ | 87 | 209 | 124 |
| | | 9 | 042 | 039 | F₄ | 85 | 209 | 67 |
| 7 | 7 | 1 | 043 | 044 | F₄ | 151 | 383 | 260 |
| | | 2 | 043 | 045 | F₄ | 151 | 383 | 254 |
| | | 3 | 044 | 046 | F₄ | 112 | 383 | 179 |
| | | 4 | 045 | 044 | F₄ | 63 | 383 | 158 |
| | | 5 | 044 | 047 | F₄ | 92 | 383 | 209 |
| | | 6 | 046 | 045 | F₄ | 154 | 383 | 195 |
| | | 7 | 045 | 047 | F₄ | 152 | 383 | 222 |

TABLE 12
Basic Information for Network Population 8 and 9

| Network | Number of | Sub | | | | Number | Number | Number of |
|---|---|---|---|---|---|---|---|---|
| Population | Sub | Population | Female | Male | Generation | of | of | Polymorphic |
| Number | Populations | Number | Parent | Parent | Analyzed | Progeny | Markers | Markers |
| 8 | 8 | 1 | 048 | 049 | F₄ | 177 | 242 | 152 |
| | | 2 | 048 | 050 | F₄ | 48 | 242 | 106 |
| | | 3 | 051 | 049 | F₄ | 151 | 227 | 116 |
| | | 4 | 052 | 049 | F₄ | 127 | 242 | 141 |
| | | 5 | 052 | 050 | F₄ | 107 | 242 | 120 |
| | | 6 | 053 | 049 | F₄ | 195 | 242 | 138 |
| | | 7 | 053 | 050 | F₄ | 55 | 242 | 117 |
| | | 8 | 054 | 049 | F₄ | 90 | 242 | 147 |
| 9 | 6 | 1 | 049 | 050 | F₄ | 45 | 232 | 170 |
| | | 2 | 055 | 049 | F₄ | 97 | 232 | 156 |
| | | 3 | 056 | 049 | F₄ | 102 | 232 | 147 |
| | | 4 | 057 | 049 | F₄ | 53 | 232 | 132 |
| | | 5 | 058 | 049 | F₄ | 156 | 232 | 164 |
| | | 6 | 051 | 049 | F₄ | 103 | 232 | 156 |

What is claimed is:

1. A method for predicting a phenotype in a plant of a predicted population, the method comprising:
(a) determining marker effects for a plurality of markers in a genotyped and phenotyped reference population with respect to a phenotype, wherein the reference population comprises:
(i) an F₂ generation produced by crossing two parental plants to produce an F₁ generation and then intercrossing, backcrossing, and/or selfing the F₁ generation; and/or making a double haploid from F₁; and/or
(ii) an F₃ or subsequent generation, wherein the F₃ or subsequent generation is produced by intercrossing, backcrossing, selfing, and/or producing double haploids from the F₂ generation and/or a subsequent generation;
(b) genotyping one or more plants of a predicted population with respect to the plurality of markers, wherein each of the one or more plants of the predicted population is a descendant of two parents and each parent has at least 80% genetic identity to at least one of the two parental plants employed to generate the reference population;
(c) summing the marker effects determined in step (a) for each of the one or more plants of the predicted population based on the genotyping of step (b); and
(d) predicting a phenotype of the one or more plants of the predicted population based on the sum of the marker effects from step (c).

2. The method of claim 1, wherein the reference population comprises a plurality of members of an F₃ or later generation generated by producing double haploids from the F₂ generation.

3. The method of claim 1, wherein the reference population is a reference network comprising a plurality of members generated by:
(i) selecting a plurality of different parental lines;
(ii) crossing the plurality of different parental lines to produce a plurality of F₁ generations;
(iii) intercrossing or backcrossing members of each F₁ generation to produce a plurality of distinct F₂ generations, and optionally singly or sequentially intercrossing, backcrossing, selfing, and/or producing double haploids from the plurality of distinct F₂ generations to produce distinct F₃ and, optionally, subsequent generations;
(iv) pooling some or all of the members of the distinct F₂, F₃, or subsequent generations to generate the reference network, wherein each member of the reference network derives its genome from two of the different parental lines.

4. The method of claim 3, wherein the reference network comprises plants derived from fewer than all possible crosses amongst the plurality of different parental lines.

5. The method of claim 4, wherein the plant of the predicted population is an F₂ or subsequent generation of a cross between two members of the plurality of different parental lines that is not included in the reference network.

6. The method of claim 3, wherein the reference network comprises plants derived from all possible crosses amongst the plurality of different parental lines.

7. The method of claim 6, wherein the plant of the predicted population is an F₂ or subsequent generation of a cross between two parents, each of which is at least 80% genetically identical to one of the plurality of different parental lines that were employed to generate the reference network.

8. The method of claim 1, wherein the reference population comprises at least 50 members, optionally at least 100 members, optionally at least 150 members, and further optionally at least 200 members.

9. The method of claim 1, wherein the determining step comprises estimating the marker effects for each of the plurality of markers by genome-wide best linear unbiased prediction (GBLUP).

10. The method of claim 1, wherein the plurality of markers are sufficient to cover the genome of the plants of the reference population such that the average interval between adjacent markers on each chromosome is less than about 10 cM, optionally less than about 5 cM, optionally less than about 2 cM, and further optionally less than about 1 cM.

11. The method of claim 1, wherein each member of the reference population, each of the one or more plants of the predicted population, or both are inbred plants or double haploids.

12. The method of claim 1, wherein the genotyping step comprising genotyping the one more plants as seeds, genotyping leaf tissue obtained from growing the one or more plants, or a combination thereof.

13. The method of claim 12, further comprising isolating the leaf tissue from the one or more plants as the one or more plants are growing in a green house.

14. The method of claim 1, wherein the genetic identity between each parent and at least one of the two parental plants employed to generate the reference population is determined by calculating a percentage of shared pre-selected markers between each of the parents and the at least one of the two parental plants employed to generate the reference population.

15. The method of claim 1, wherein predicting step (d) comprises employing a linear model for genome-wide best linear unbiased prediction (GBLUP) as set forth in Equation (4):
$${y}_{i} = μ + {\sum }_{j = 1}^{m} ( {z}_{ij} {g}_{j} ) + {e}_{i} , \qquad ( 4 )$$
wherein:
(i) yᵢ is the phenotypic BLUP of the line i, μ is the overall mean, zᵢⱼ is the genotype of the marker j for the line i, gⱼ is the effect of the marker j, and eᵢ the residual following eᵢ˜N(0, σₑ²);
(ii) μ is assumed to be a fixed effect and gⱼ is assumed to be a random effect following a normal distribution gⱼ˜N(0, σ_{gj}²);
(iii) each marker is assumed to have an equal genetic variance expressed by Equation (4a):
σ_{gj}²=σ_{g}²/m (4a),
with m the total number of markers used;
(iv) a variance-covariance matrix V for the phenotype y is expressed by Equation (4b):
$$V = {\sum }_{j = 1}^{m} ( {Z}_{j} {Z}_{j}^{T} {σ}_{gj}^{2} ) + {I}_{( n \times n )} {σ}_{e}^{2} \qquad ( 4 b )$$
wherein Zⱼ is a vector of genotypic scores of the marker j across n individuals in a population and I_{(n×n)} is an identity matrix with diagonal elements 1 and others 0;
(v) overall mean p, a fixed effect, is estimated as set forth in Equation (4c):
\hat{μ}=(X^{T}V⁻¹X)⁻¹X^{T}V⁻y (4c)
with X a vector of ones, and ĝⱼ, the effect of the marker j, is calculated as set forth in Equation (4d):
ĝⱼ=σ_{gj}²ZⱼV⁻¹(y−X\hat{μ}) (4d).

16. The method of claim 15, wherein predicting step (d) is performed by a suitably-programmed computer.

17. The method of claim 1, further comprising selecting one or more of the one or more plants of the predicted population that are predicted to have the phenotype of interest.

18. The method of claim 17, wherein the selecting considers several traits of interest, and a multi-trait selection index is calculated for an individual in the predicted population.

19. The method of claim 18, wherein the multi-trait selection index is calculated for a progeny individual in the predicted population using Equation (6):
$${I}_{i} = {\sum }_{j = 1}^{t} [ {w}_{j} \frac{{\hat{y}}_{i}^{j} - Min ( {\hat{y}}^{j} )}{Max ( {\hat{y}}^{j} ) - Min ( {\hat{y}}^{j} )} ] \qquad ( 6 )$$
and further wherein:
(i) Iᵢ is a multi-trait selection index for the progeny i;
(ii) wⱼ is a weight ranging from 0 to 1 for trait j used for measuring the relative importance of the trait j;
(iii) ŷᵢ^{j} is a predicted phenotype of the trait j (j=1, 2, . . . , t) in the progeny;
(iv) Min(ŷ^{j}) is a minimum value of the predicted phenotypes of the trait j in all the progeny in the predicted population; and
(v) Max(ŷ^{j}) is a maximum value of the predicted phenotypes of the trait j in all the progeny in the predicted population.

20. The method of claim 19, wherein the multi-trait selection index calculation is performed by a suitably-programmed computer.

21. The method of claim 16, further comprising growing one or more of the one or more plants of the predicted population that are predicted to have the phenotype of interest in tissue culture or by planting.

22. A method for predicting a phenotype in a plant of a predicted population, the method comprising:
(a) determining marker effects for a plurality of markers in a genotyped and phenotyped reference population, wherein the reference population comprises a linkage disequilibrium (LD) panel;
(b) genotyping one or more plants of the predicted population with respect to the plurality of markers, wherein each of the one or more plants of the predicted population is a descendant of two parents, each of which is at least 80% genetically identical to a member of the reference population;
(c) summing the marker effects for each of the one or more plants of the predicted population based on the genotyping of step (b); and
(d) predicting the phenotype of the one or more plants of the predicted population based on the marker effects summed in step (c).

23. The method of claim 22, wherein each of the one or more plant of the predicted population is an F₁ generation plant produced by crossing two members of the reference population or is an F₂ or subsequent generation plant produced by singly or multiply intercrossing, backcrossing, selfing, and/or producing double haploids from the F₁ generation plant or any subsequent generation thereof.

24. The method of claim 22, wherein each of the plants of the predicted population is an F₁ generation plant produced by crossing two parental plants, each of which is at least 80% genetically identical to a member of the reference population.

25. The method of claim 22, wherein the reference population comprises at least 50 members, optionally at least 100 members, optionally at least 150 members, optionally at least 200 members, and further optionally at least 250 members.

26. The method of claim 22, wherein the determining step comprises calculating the marker effects for each of the plurality of markers by genome-wide best linear unbiased prediction (GBLUP).

27. The method of claim 22, wherein the plurality of markers are sufficient to cover the genome of the plants of the reference population such that the average interval between adjacent markers on each chromosome is less than about 1 cM, optionally less than about 0.5 cM, and optionally less than about 0.1 cM.

28. The method of claim 22, wherein each member of the reference population, each of the one or more plants of the predicted population, or both are inbred plants or double haploids.

29. The method of claim 22, further comprising identifying an core set of markers using a preselected significance level determined by a method of combining cross validations, single marker regression, and GBLUP and employing the core set of markers in summing step (c).

30. The method of claim 22, further comprising selecting one or more of the one or more plants of the predicted population that are predicted to have the phenotype of interest and reproducing the same in tissue culture or by planting.

31. A method for generating a plant with a phenotype of interest, the method comprising:
(a) determining marker effects for a plurality of markers in a genotyped and phenotyped reference population, wherein the reference population comprises:
(i) an F₂ generation produced by crossing two parental plants to produce an F₁ generation and then intercrossing, backcrossing, and/or selfing the F₁ generation; and/or
(ii) an F₃ or subsequent generation, wherein the F₃ or subsequent generation is produced by intercrossing, backcrossing, selfing, and/or producing double haploids from the F₂ generation and/or a subsequent generation; and/or
(iii) a reference network comprising a plurality of members generated by:
(1) selecting a plurality of different parental lines;
(2) crossing the plurality of different parental lines to produce a plurality of F₁ generations;
(3) intercrossing, backcrossing, and/or selfing the F₁ generation; and/or making a double haploid from F₁ to produce a plurality of distinct F₂ generations, and optionally singly or sequentially intercrossing, backcrossing, selfing, and/or producing double haploids from the plurality of distinct F₂ generations to produce distinct F₃ and, optionally, subsequent generations;
(4) pooling some or all of the members of the distinct F₂, F₃, or subsequent generations to generate the reference network, wherein each member of the reference network derives its genome from two of the parental lines; and/or
(iv) a linkage disequilibrium (LD) panel;
(b) genotyping one or more plants of a predicted population with respect to the plurality of markers, wherein the each of the one or more plants of the predicted population is a descendant of two parents each of which is at least 80% genetically identical to at least one of the two plants that comprise or where employed to generate the reference population;
(c) summing the marker effects for each of the one or more plants of the predicted population based on the genotype determined in step (b) to generate a genetic score for each of the one or more plants of the predicted population;
(d) predicting phenotypes of the one or more plants of the predicted population based on the genetic scores generated in step (c);
(e) selecting one or more of the one or more plants of the predicted population based on the predicting step that are predicted to have a phenotype of interest, and
(f) growing the selected one or more plants of the predicted population,
wherein a plant with a phenotype of interest is generated.

32. The method of claim 31, wherein the selecting step comprises selecting those plants of the predicted population that have a genetic score that exceeds a pre-selected threshold.

33. A method for estimating genetic similarity between a first and a second population, the method comprising:
(a) providing a first and a second population, wherein:
(i) the first population comprises individuals that are F₂ or subsequent generation progeny produced by crossing a first parent and a second parent to produce a first F₁ generation, and then intercrossing, backcrossing, selfing, and/or producing double haploids from the first F₁ generation to produce the F₂ generation, and optionally, further intercrossing, backcrossing, selfing, and/or producing double haploids from the F₂ generation and any subsequent generations to produce the first population; and
(ii) the second population comprises individuals that are F₂ or subsequent generation progeny produced by crossing a third parent and a fourth parent to produce a second F₁ generation, and then intercrossing, backcrossing, selfing, and/or producing double haploids from the second F₁ generation to produce the F₂ generation, and optionally, further intercrossing, backcrossing, selfing, and/or producing double haploids from the F₂ generation and any subsequent generations to produce the second population;
(b) genotyping the first, second, third, and fourth parents with respect to a plurality of pre-determined markers;
(c) calculating first, second, third, and fourth percent genetic similarities, wherein:
(i) the first percent genetic similarity is the percentage of allele sharing across all of the pre-determined markers of the first parent with respect to the third parent;
(ii) the second percent genetic similarity is the percentage of allele sharing across all of the pre-determined markers of the first parent with respect to the fourth parent;
(iii) the third percent genetic similarity is the percentage of allele sharing across all of the pre-determined markers of the second parent with respect to the third parent; and
(iv) the fourth percent genetic similarity is the percentage of allele sharing across all of the pre-determined markers of the second parent with respect to the fourth parent;
(d) determining a first mean percentage genetic similarity comprising the mean percentage genetic similarity of the first percent genetic similarity and the third percent genetic similarity;
(e) determining a second mean percentage genetic similarity comprising the mean percentage genetic similarity of the second percent genetic similarity and the fourth percent genetic similarity; and
(f) selecting the greater of the first mean percentage genetic similarity and the second mean percentage genetic similarity, wherein the greater of the two mean percentage genetic similarities provides an estimate of the genetic similarity between a first and a second population.

34. The method of claim 33, wherein the first population and the second population consist of F₄ progeny produced by selfing F₁, F₂, and F₃ individuals from the first F₁ population and the second F₁ population, respectively.

35. The method of claim 33, wherein the plurality of pre-determined markers span substantially the entire genomes of the first and second populations.
